(19) United States
(12) Patent Application Publication
(10) Pub. No.: US 2024/0207239 A1
(43) Pub. Date: Jun. 27, 2024

(54) COMPOSITION AND PREPARATION METHOD THEREFOR

(71) Applicant: SUNSHINE LAKE PHARMA CO., LTD., Dongguan, Guangdong (CN)

(72) Inventors: Yingchao XU, Dongguan (CN); Xue LI, Dongguan (CN); Lirong WANG, Dongguan (CN); Tianxiang SHI, Dongguan (CN); Xin HUANG, Dongguan (CN); Jinsong YOU, Dongguan (CN); Fangfang HUANG, Dongguan (CN); Qian WANG, Dongguan (CN)

(73) Assignee: SUNSHINE LAKE PHARMA CO., LTD., Dongguan, Guangdong (CN)

(21) Appl. No.: 18/574,896

(22) PCT Filed: Jan. 26, 2022
(86) PCT No.: PCT/CN2022/073952

§ 371 Date: Dec. 28, 2023

(30) Foreign Application Priority Data
Feb. 6, 2021 (CN) 202110165171.X

(51) Int. Cl. A61K 31/4439; A61K 9/16; A61K 9/51

(52) U.S. Cl. CPC A61K 31/4439; A61K 9/1617; A61K 9/1623; A61K 9/1652; A61K 9/1676; A61K 9/5161; A61K 9/5192

(57) ABSTRACT

A composition and a preparation method therefor, the composition including an active ingredient and a stabilizer, wherein the stabilizer includes an ionic surfactant and a spatial stabilizer. The composition has advantages, such as a high dissolution rate, a fast dissolution speed, a high particle size stability and a high bioavailability, as well as a small tablet weight, granule weight or preparation volume. The preparation method has advantages such as simple operation and being environmentally friendly.

### CROSS-REFERENCE TO RELATED APPLICATION

This application claims priority to Chinese Patent Application CN 202110165171.X filed on Feb. 6, 2021, incorporated herein by reference in its entirety.

### FIELD OF THE INVENTION

The present invention relates to the field of pharmaceutical formulations, and in particular relates to a kind of apalutamide nanocrystalline composition and preparation method thereof.

### BACKGROUND ART

The chemical name of apalutamide is 4-[7-(6-cyano-5-trifluoromethylpyridine-3-yl)-8-oxo-6-thioxo-5, 7-diazaspiro[3. 4] octan-5-yl]-2-fluoro-N-methylbenzamide, the chemical structure of which is shown below:

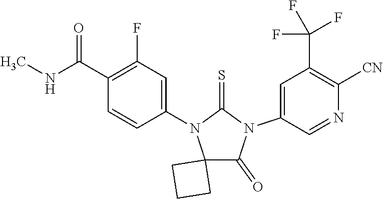 [SMILES: CNC(=O)c1ccc(N2C(=S)N(c3cnc(C#N)c(C(F)(F)F)c3)C(=O)C23CCC3)cc1F]

Apalutamide is almost insoluble in aqueous medium in the range of pH 1-12, and is a BCS Class II drug, which is a poorly soluble drug, if it is not specially treated, but is directly prepared into an oral solid preparation, such as tablets or capsules, the drug dissolution rate is slow and incomplete, and the bioavailability is low, thus limiting the efficacy of the oral preparation. At present, the marketed apalutamide tablets (trade name Erleada®) are 60 mg and taken once a day, but 4 tablets are taken at a time for a total of 240 mg specifications. Due to the use of solid dispersion technology for solubilization in the original research, coupled with its own relatively large specifications and high proportion of carriers, the final product has a large tablet weight, with each 60 mg tablet weighing more than 720 mg, and 4 tablets need to be taken at a time, which restricts the medication of elderly patients and other people who are inconvenient to swallow, and seriously reduces the compliance of patients.

At present, the disclosed technology for solubilization of apalutamide preparations is the solubilization of solid dispersions. In the patents CN106999430A, CN106999431A and CN106999432A, the solid dispersion of apalutamide was prepared by spray drying technology using dichloromethane, methanol and/or acetone as solvents, and then other excipients were further added to prepare tablets. Because the solubilization of the solid dispersion requires a high proportion of carriers, and the solid dispersion materials prepared by the spray drying process are fluffy and have poor fluidity, so the prepared solid dispersion also needs to be prepared by dry granulation process to obtain particles with good fluidity, and then a large number of excipients are added to press into tablets, resulting in low drug loading. The weight of each tablet of 60 mg of apalutamide tablets is more than 700 mg, and the weight of 120 mg of apalutamide tablets is up to 1400 mg (see example 6.2 of CN106999431A, example 3.2 of CN106999430A, example 3.2 of CN106999432A). The tablets are heavy, which greatly increases the difficulty of swallowing and seriously reduces the patient's compliance, and the spray drying process in this patent uses a large amount of organic solvents, which increases the production risk and is not conducive to environmental protection.

In addition, the solid dispersion of apalutamide is prepared by hot melt extrusion technology in the patents CN106999430A, CN106999431A and CN106999432A, and other excipients are further added to prepare tablets. Apalutamide has a high melting point of 194-196° C., which leads to high operating temperature of hot melt extrusion, which easily leads to degradation of active substances, increases stability risks, and has high requirements for the workshop cooling system during the extrusion process.

Micron-sized drug particles of drug nanocrystals are dispersed or crystallized by milling, reducing the particle size to sub-micron (100-1000 nm) or even nanon (1-100 nm). Drug nanocrystals do not require special carrier materials, and the introduction of stable inactive substances is less, and most of them are inactive substances with good safety, which are not easy to cause toxic side effects.

However, on the one hand, the drug delivery system of drug nanocrystals is a thermodynamic and kinetic unstable system, which is prone to coalescence and crystal growth, resulting in a slower dissolution rate. On the other hand, whether the nanocrystals are still in a nano state after solidification and redispersion is also an urgent problem to be solved. The wettability, saturation solubility and dissolution speed of the drug can be improved only when the nanocrystalline solidified preparation is still in a nano state when the gastrointestinal fluid is redispersed after taking it. If it is agglomerated and coalescence after reconstitution, it will not achieve the technical effect of improving solubility and dissolution speed. Regardless of the use of freeze drying, spray drying, fluidized bed drying or vacuum drying and other curing methods, due to the loss of water during the curing process of nanocrystalline suspension, various stresses will be generated, resulting in “curing damage”, which will lead to irreversible aggregation of drug nanoparticles, and then lead to agglomeration and coalescence after reconstitution, which cannot improve the solubility and dissolution speed of the technical effect.

In short, the low solubility of apalutamide leads to low bioavailability, so the bioavailability can be significantly improved after solubilization, while the existing solubilization technologies of apalutamide are solid dispersion technologies. However, the solubilization of solid dispersion technology, whether spray drying technology or hot melt extrusion technology, requires at least several times the carrier of apalutamide, resulting in too low drug loading, which in turn leads to in a large tablet weight, granular weight or volume of apalutamide preparations, which restricts the medication of elderly patients and other people who are inconvenient to swallow, and seriously reduces the compliance of patients. However, the use of nanocrystalline technology has problems such as easy coalescence, crystal growth, slow dissolution speed, and nanocrystals are not in a nano state when solidified and redispersed.

Therefore, it is still necessary to develop an apalutamide composition with high drug loading, small tablet weight, granule weight or preparation volume, fast dissolution rate and high bioavailability.

### SUMMARY

### Summary of the Invention

An object of the present invention is to provide an apalutamide composition that is not easy to coalescence, has a high drug loading, small tablet weight, granule weight or preparation volume, a fast dissolution rate, dissolution rate stability and a high bioavailability. Thus, the present invention provides the following technical scheme.

In the first aspect, the present invention provides a composition comprising a nanocrystalline composition, the nanocrystalline composition comprises an active ingredient and a stabilizer, the active ingredient is apalutamide or pharmacologically acceptable salt thereof. The stabilizer can comprise an ionic surfactant and a spatial stabilizer. The ionic surfactant is preferably sodium dodecyl sulfate. The spatial stabilizer is preferably povidone K17 or hydroxypropyl cellulose. The nanocrystalline composition is conducive to improving the particle size stability of the composition, reducing the tablet weight, granule weight or preparation, improving the dissolution speed and the dissolution stability, and improving the bioavailability. The spatial stabilizer is preferably hydroxypropyl cellulose, and compared with other spatial stabilizers, the use of hydroxypropyl cellulose is more conducive to improving the dissolution rate and in vivo bioavailability of the composition.

In the second aspect, the present invention provides a method for preparing the composition described in the first aspect.

### DETAILED DESCRIPTION OF THE INVENTION

In order to solve the problems existing in the prior art, the present invention provides a composition and a preparation method thereof.

The present invention finds a large number of stabilizers in the process of screening, using a single kind of stabilizer to prepare apalutamide nanocrystals, it is difficult to obtain nano-scale nanoparticles, and even if obtained, coalescence is easy to occur in the process of long-term placement, resulting in the slowing down of the dissolution rate. Therefore, the present invention obtains an apalutamide composition that is not easy to coalesce, has high drug loading, tablet weight, granule weight or preparation volume is small, fast dissolution rate, stable dissolution rate and high bioavailability by screening suitable stabilizer types, quantity and proportion, etc.

Thus, in the first aspect, the present invention provides a composition.

A composition comprises a nanocrystalline composition, the nanocrystalline composition comprises an active ingredient and a stabilizer, the active ingredient is apalutamide or pharmacologically acceptable salt thereof.

The stabilizer can comprise an ionic surfactant and a spatial stabilizer.

The ionic surfactant can comprise at least one of sodium dodecyl sulfate, sodium dodecyl sulfonate, sodium dioctyl sulfosuccinate, benzethonium chloride, sodium docusate, lecithin, and arginine hydrochloride. In some preferred embodiments, the ionic surfactant is sodium dodecyl sulfate.

The spatial stabilizer can comprise at least one of hydroxypropyl cellulose, polyvinylpyrrolidone (povidone), vinylpyrrolidone-vinyl acetate copolymer, polyvinyl alcohol, hydroxypropyl methylcellulose, hydroxypropyl methylcellulose acetate succinate, hydroxymethyl cellulose, chitosan, gelatin, carrageenan and cyclodextrin. In some preferred embodiments, the spatial stabilizer is selected from at least one of povidone K17, hydroxypropyl cellulose, vinylpyrrolidone-vinyl acetate copolymer, polyvinyl alcohol, hydroxypropyl methylcellulose (e.g., hydroxypropyl methylcellulose E5 or hydroxypropyl methylcellulose E3) and hydroxypropyl methylcellulose acetate succinate; Compared with other spatial stabilizers, the use of at least one of povidone K17, hydroxypropyl cellulose, vinylpyrrolidone-vinyl acetate copolymer, polyvinyl alcohol, hydroxypropyl methylcellulose (e.g., hydroxypropyl methylcellulose E5 or hydroxypropyl methylcellulose E3) and hydroxypropyl methylcellulose acetate succinate is more beneficial for increasing dissolution rate and dissolution speed of the composition. In some embodiments, the spatial stabilizer is povidone, and the povidone is preferably povidone K17; compared with other types of povidone, the use of povidone K17 is more conducive to obtaining a nanocrystalline composition with good particle size stability during placement, and is more conducive to improving the dissolution rate and the stability of the particle size of the nanocrystalline composition after curing and reconstitution. In some embodiments, the spatial stabilizer is hydroxypropyl cellulose, and the use of hydroxypropyl cellulose is conducive to obtaining a nanocrystalline composition with good particle size stability during placement, and is more conducive to improving the dissolution rate and the stability of the particle size of the nanocrystalline composition after curing and reconstitution. In some more preferred embodiments, the spatial stabilizer is at least one of hydroxypropyl cellulose, vinylpyrrolidone-vinyl acetate copolymer, polyvinyl alcohol, hydroxypropyl methylcellulose (e.g., hydroxypropyl methylcellulose E5 or hydroxypropyl methylcellulose E3) and hydroxypropyl methylcellulose acetate succinate, which is more conducive to improving the dissolution rate and in vivo bioavailability of the composition.

The active ingredient is based on apalutamide, and the mass ratio of the ionic surfactant to the active ingredient can be 1:200-1:2. In some embodiments, the active ingredient is based on apalutamide, and the mass ratio of the ionic surfactant to the active ingredient is 1:100-1:2. In some embodiments, the active ingredient is based on apalutamide, and the mass ratio of the ionic surfactant to the active ingredient is 1:50-1:2. In some embodiments, the active ingredient is based on apalutamide, and the mass ratio of the ionic surfactant to the active ingredient is 1:20-3:10.

The active ingredient is based on apalutamide, and the mass ratio of the spatial stabilizer to the active ingredient can be 1:30-2:1. In some embodiments, the mass ratio of the spatial stabilizer to the active ingredient is 1:20-1:1. In some embodiments, the mass ratio of the spatial stabilizer to the active ingredient is 1:15-1:2. In some preferred embodiments, the mass ratio of the spatial stabilizer to the active ingredient is 1:10-1:2. In some embodiments, the mass ratio of the spatial stabilizer to the active ingredient can be 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1 or 2:1.

The particle size D90 of the nanocrystalline composition can be 1 nm-1000 nm (e.g., about 100 nm, 200 nm, 300 nm, 400 nm, 500 nm, 600 nm, 700 nm, 800 nm, 900 nm or 1000 nm). The particle size D90 of the nanocrystalline composition can be about 1000 nm or less (e.g., about 100 nm or less, about 200 nm or less, about 300 nm or less, about 400 nm or less, about 500 nm or less, about 600 nm or less, about 700 nm or less, about 800 nm or less, about 900 nm or less, or about 1000 nm or less). In some embodiments, the particle size D90 of the nanocrystalline composition is 1 nm-800 nm. In some embodiments, the particle size D90 of the nanocrystalline composition is 1 nm-700 nm. In some embodiments, the particle size D90 of the nanocrystalline composition is 1 nm-600 nm. In some embodiments, the particle size D90 of the nanocrystalline composition is 1 nm-500 nm. In some embodiments, the particle size D90 of the nanocrystalline composition is 1 nm-400 nm. In some embodiments, the particle size D90 of the nanocrystalline composition is 10 nm-300 nm. In some embodiments, the particle size D90 of the nanocrystalline composition is 10 nm-200 nm. In some embodiments, the particle size D90 of the nanocrystalline composition is 50 nm-100 nm. In some embodiments, the particle size D90 of the nanocrystalline composition is 200 nm-700 nm. In some embodiments, the particle size D90 of the nanocrystalline composition is 300 nm-600 nm.

The particle size D50 of the nanocrystalline composition can be about 900 nm or less. In some embodiments, the particle size D50 of the nanocrystalline composition is about 800 nm or less. In some embodiments, the particle size D50 of the nanocrystalline composition is about 700 nm or less. In some embodiments, the particle size D50 of the nanocrystalline composition is about 600 nm or less. In some embodiments, the particle size D50 of the nanocrystalline composition is about 500 nm or less. In some embodiments, the particle size D50 of the nanocrystalline composition is about 400 nm or less.

The composition can also include a protective agent.

The protective agent can comprise at least one of mannitol, lactose, fructose, glycine, glucose, sucrose, maltose, trehalose, sorbitol, xylitol, polydextrose, fructooligosaccharides, malto-oligosaccharides, galacto-oligosaccharides, dextrin, gum arabic, microcrystalline cellulose, anhydrous calcium hydrogen phosphate, pregelatinized starch and corn starch. In some embodiments, the protective agent preferably comprises at least one of mannitol, lactose, fructose, glycine, glucose, maltose, trehalose, xylitol, polydextrose, fructooligosaccharides, malto-oligosaccharides, galacto-oligosaccharides, dextrin, and gum arabic. In some embodiments, the protective agent is preferably mannitol. In some embodiments, the protective agent is preferably microcrystalline cellulose.

The active ingredient is based on apalutamide, and the mass ratio of the protective agent to the active ingredient can be 8:1-1:8 (e.g., 8:1, 7:1, 6:1, 5:1, 4:1, 3:1, 2:1, 1:1, 1:2, 1:3, 1:4, 1:5, 1:6, 1:7 or 1:8). In some embodiments, the mass ratio of the protective agent to the active ingredient is 8:1-1:4. In some embodiments, the mass ratio of the protective agent to the active ingredient is 8:1-1:2. In some embodiments, the mass ratio of the protective agent to the active ingredient is 4:1-1:6. In some embodiments, the mass ratio of the protective agent to the active ingredient is 4:1-1:2.

The active ingredient is based on apalutamide, and the content of the active ingredient in the non-volatile components of the nanocrystalline composition can be 10 wt %-95 wt %.

The nanocrystalline composition can also comprise water.

The non-volatile components of the nanocrystalline composition refer to other components other than water, such as active ingredients and stabilizers, etc.

The active ingredient is based on apalutamide, and the content of the active ingredient in the non-volatile components of the composition can be 10 wt %-40 wt %. In some embodiments, the active ingredient is based on apalutamide, and the content of the active ingredient in the non-volatile components of the composition can be 15 wt %-25 wt %.

The non-volatile components of the composition refer to other components other than water, such as active ingredients, stabilizers and protective agents, etc.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient is 1:30-2:1.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient is 1:15-2:1 or 1:10-2:1.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient can be 1:30-2:1; the nanocrystalline composition also comprises a protective agent, and the mass ratio of the protective agent to the active ingredient can be 8:1-1:8.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient can be 1:15-2:1 or 1:10-2:1; the nanocrystalline composition also comprises a protective agent, and the mass ratio of the protective agent to the active ingredient is 8:1-1:4 or 8:1-1:2.

In some embodiments of the present invention, the composition comprises a nanocrystalline composition, the nanocrystalline composition comprises an active ingredient and a stabilizer, the active ingredient is apalutamide or a pharmaceutically acceptable salt thereof; the particle size D90 of the nanocrystalline composition is 1 nm-1000 nm. In some embodiments of the present invention, the composition comprises a nanocrystalline composition, the nanocrystalline composition comprises an active ingredient and a stabilizer, the active ingredient is apalutamide or a pharmaceutically acceptable salt thereof; the particle size D50 of the nanocrystalline composition is 700 nm or less.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient can be 1:10-2:1; the composition also comprises a protective agent, and the mass ratio of the protective agent to the active ingredient can be 8:1-1:2; the particle size D90 of the nanocrystalline composition is 1000 nm or less.

In some embodiments of the present invention, the composition comprises a nanocrystalline composition, the nanocrystalline composition comprises an active ingredient and a stabilizer, the active ingredient is apalutamide or a pharmaceutically acceptable salt thereof, and the stabilizer comprises an ionic surfactant and a spatial stabilizer, the active ingredient is based on apalutamide, and the mass ratio of the ionic surfactant to the active ingredient is 1:200-1:2, the mass ratio of the spatial stabilizer to the active ingredient can be 1:10-2:1; the composition also comprises a protective agent, and the mass ratio of the protective agent to the active ingredient can be 8: 1-1:2; the particle size D50 of the nanocrystalline composition is 700 nm or less.

The nanocrystalline composition can be prepared by at least one method selected from precipitation method, emulsification method, high-pressure homogenization method and medium milling method.

In some embodiments, the nanocrystalline composition is prepared by a medium milling method.

In the second aspect, the present invention provides a method for preparing the aforesaid composition.

A method for preparing the aforesaid nanocrystalline composition, comprising:
(1) Stabilizer and apalutamide are mixed with water to obtain a suspension;
(2) The suspension is mixed with a milling medium and milled to obtain a nanocrystalline composition.

The preparation method of the aforesaid composition comprises the following steps:
(1) Stabilizer and apalutamide are mixed with water to obtain a suspension;
(2) The suspension is mixed with a milling medium and milled to obtain a nanocrystalline composition;
(3) The nanocrystalline composition is optionally added with pharmaceutically acceptable excipients, mixed, to obtain a composition; or the nanocrystalline composition is added with a protective agent, cured by a curing process, optionally added with at least one of filler, disintegrant, glidant or lubricant, mixed, to obtain a composition.

The composition can be an oral nanosuspension or an oral solid preparation.

The solid content of the suspension can be 2 wt %-30 wt %.

The pharmaceutically acceptable excipients can comprise flavor correcters, pH adjusters or preservatives.

The curing process can comprise spray drying, fluidized bed spray drying or freeze drying. In some embodiments, the curing process is spray drying or freeze-drying, and the protective agent comprises at least one of mannitol, lactose, fructose, glycine, glucose, maltose, trehalose, xylitol, polydextrose, fructooligosaccharides, malto-oligosaccharides, galacto-oligosaccharides, dextrin, and gum arabic. In some embodiments, the curing process is spray drying or freeze drying, and the protective agent is mannitol.

In some embodiments, the curing process is fluidized bed spray drying, and the protective agent comprises at least one of mannitol, lactose, fructose, glycine, glucose, sucrose, maltose, trehalose, sorbitol, xylitol, polydextrose, fructooligosaccharides, malto-oligosaccharides, galacto-oligosaccharides, dextrin, gum arabic, microcrystalline cellulose, anhydrous calcium hydrogen phosphate, pregelatinized starch and corn starch. In some embodiments, the curing process is fluidized bed spray drying, the protective agent is mannitol, or the protective agent is microcrystalline cellulose.

The filler can comprise at least one of microcrystalline cellulose, mannitol, lactose, starch, corn starch, calcium hydrogen phosphate hydrates, calcium hydrogen phosphate, monobasic calcium phosphate, magnesium carbonate, calcium carbonate, purified sucrose and glucose.

The disintegrant can comprise at least one of corn starch, starch, crystalline cellulose, calcium carboxymethylcellulose, sodium carboxymethylcellulose, croscarmellose sodium, light anhydrous silicic acid, calcium silicate, low-substituted hydroxypropyl cellulose, partial pregelling starch, sodium carboxymethyl starch, agar powder, polyvinyl polypyrrolidone, synthetic aluminum silicate, sucrose fatty acid ester, lactose hydrate, D-mannitol, anhydrous citric acid, potassium chloride, sodium chloride, magnesium chloride, potassium dihydrogen phosphate, sodium bicarbonate, potassium hydrogen phosphate, potassium sulfate, sodium sulfate, sodium carbonate and calcium chloride.

The glidant can be micronized silica gel.

The lubricant can comprise at least one of magnesium stearate, calcium stearate, sucrose fatty acid ester, sodium stearyl fumarate, polyethylene glycol, talc and stearic acid.

In some embodiments of the present invention, a method for preparing the aforesaid composition comprises the following steps:
(1) Spatial stabilizer, ionic surfactant and apalutamide are mixed with water to obtain a suspension;
(2) The suspension is mixed with a milling medium and milled to obtain a nanocrystalline composition;
(3) The nanocrystalline composition is optionally added with flavor corrector, pH adjuster or preservative, mixed to obtain a composition; the composition is an oral nanosuspension.

In some embodiments of the present invention, a method for preparing the aforesaid composition comprises the following steps:
(1) Spatial stabilizer, ionic surfactant and apalutamide are mixed with water to obtain a suspension;
(2) The suspension is mixed with a milling medium and milled to obtain a nanocrystalline composition;
(3) A protective agent is added the nanocrystalline composition, curing through a curing process, the curing process comprises spray drying, fluidized bed granulation or freeze drying, and then at least one of filler, disintegrant, glidant or lubricant is optionally added and mixed to obtain a composition.

The composition can be compressed to obtain tablets.

The composition can be loaded into capsule shell to obtain capsules.

### Beneficial Effects

Compared with the prior art, the present invention has a beneficial effect including at least one of the following:

(1) Compared with the apalutamide nanocrystalline tablets prepared with polyvinyl caprolactam-polyvinyl acetate-polyethylene glycol graft copolymer, Tween-80, poloxamer, polyethylene glycol 6000 as a spatial stabilizer, the apalutamide nanocrystalline tablets prepared by povidone K17, hydroxypropyl cellulose, vinylpyrrolidone-vinyl acetate copolymer, polyvinyl alcohol, hydroxypropyl methylcellulose (e.g., hydroxypropyl methylcellulose E5 or hydroxypropyl methylcellulose E3), or hydroxypropyl methylcellulose acetate succinate as the spatial stabilizerhave higher dissolution rate and faster dissolution speed in pH 6.8 media; so at least one of povidone K17, hydroxypropyl cellulose, vinylpyrrolidone-vinyl acetate copolymer, polyvinyl alcohol, hydroxypropyl methylcellulose (e.g., hydroxypropyl methylcellulose E5 or hydroxypropyl methylcellulose E3) and hydroxypropyl methylcellulose acetate succinate is preferred as the spatial stabilizer.

(2) Povidone models are used as K12, K17 and K30, that is, povidone with an average molecular weight of about 2500, 10000 and 50000, and the preferred povidone model is K17, which is conducive to the particle size stability of the milling composition. The spatial stabilizer can be preferably povidone K17 or hydroxypropyl cellulose, and compared with other types of povidone, the use of povidone K17 or hydroxypropyl cellulose is more conducive to obtaining a nanocrystalline composition with good particle size stability in the placing process, and is more conducive to improving the dissolution rate and the stability of the particle size after the nanocrystalline composition is cured and reconstituted.

(3) the spatial stabilizer is preferably at least one of hydroxypropyl cellulose, vinylpyrrolidone-vinyl acetate copolymer, polyvinyl alcohol, hydroxypropyl methyl cellulose (e.g., hydroxypropyl methyl cellulose E5 or hydroxypropyl methylcellulose E3) and hydroxypropyl methylcellulose acetate succinate, and is more conducive to improving the dissolution rate and in vivo bioavailability of the composition than other spatial stabilizers.

(4) The weight ratio of spatial stabilizer to apalutamide can greatly improve the dissolution rate of apalutamide nanocrystalline tablets in the range of 1:15˜1:2, and the weight ratio of spatial stabilizer to apalutamide is preferably 1:10˜1:2.

(5) In the case of spatial stabilizer (such as hydroxypropyl cellulose) in the composition of apalutamide, the solubilization effect of adding ionic surfactant is far better than that of adding other types of surfactants, among which the solubilization effect of sodium dodecyl sulfate or sodium docusate is better, and the solubilization effect of lecithin as surfactant can also be significantly improved.

(6) When the weight ratio of ionic surfactant to apalutamide is in the range of 1:200-1:2, the obtained apalutamide nanocrystalline tablets has high dissolution rate and fast dissolution speed in pH 6.8 medium, and can avoid a large number of bubbles in the milling process and avoid too much ionic surfactant from harming the human body.

(7) The weight ratio of the protective agent to apalutamide is preferably 8:1-1:2, which can not only achieve the technical effect of high dissolution rate and fast dissolution speed, but also reduce the weight of the tablets and improve the physical stability of the particle size of the apalutamide nanocrystalline composition.

(8) Milling apalutamide composition is beneficial to improve the bioavailability of apalutamide.

(9) Compared with the reference listed drug, the present invention greatly improves the drug loading, greatly reduces the weight of the preparation of the same specification, is more conducive to the patient's taking, and improves the use compliance.

(10) The preparation method of the composition provided by the present invention has advantages such as simple operation, using no organic solvent, and being environmentally friendly.

### DESCRIPTION OF THE DRAWINGS

FIG. 1 is the drug time curve of the experimental group and the control group in Example 31, wherein the horizontal axis is time, the unit is hour, and the vertical axis is the blood drug concentration, and the unit is ng/mL.

### DEFINITION OF TERMS

In the present invention, “Soluplus” means “polyvinyl caprolactam-polyvinyl acetate-polyethylene glycol graft copolymer”.

When the term “composition” is described separately, the term “composition” means a nanocrystalline composition or a combination of nanocrystalline compositions and other components.

The term “D90” means the particle size of a sample when the cumulative particle size distribution reaches 90%. Its physical meaning is that 90% of the particles are smaller than it, for example, “D90 is not larger than 100 μm” means “90% of the particles are not larger than 100 μm”. D10 means the particle size corresponding to the cumulative particle size distribution of a sample reaching 10%, and D50 means the particle size corresponding to the cumulative particle size distribution of a sample reaching 50%.

In the present invention, the solid content means the mass percentage of non-volatiles in a solution, suspension or emulsion.

The term “comprise” and its variations such as “comprises” and “comprising” should be understood as open-ended, i.e. “comprise but not limited to”. When used to define compositions and methods, “basically consists of . . . ” or its grammatical variants shall indicate the exclusion of other elements of any importance to the composition and the method of preparation, but not factors that have no substantial effect on the composition and the method of preparation. “Consists of . . . ” or its grammatical variants shall indicate the exclusion of elements not explicitly enumerated. The embodiments defined by each of these transitional terms are within the scope of the present invention. For example, when a formulation is described as comprising components A, B, and C, the formulation is essentially composed of A, B, and C, and the formulation consists of A, B, and C, independently within the scope of the present invention.

The singular forms “a,” “an,” and “the” comprise the plurals unless the context clearly dictates otherwise. For example, a reference to “stabilizer” comprises one or more stabilizers.

In the foregoing of the present invention, all figures disclosed herein are approximate, regardless of whether the words “approximately” or “about” are used.

Based on the published figures, there may be a difference of less than ±10% in the value of each number, or a difference that is considered reasonable by those in the field, such as ±1%, ±2%, ±3%, ±4%, or ±5%.

“Pharmacologically acceptable” means a substance or compound that, within the limits of sufficient medical judgment, is suitable for contact with human and lower animal tissues without unsuitable toxicity, irritation, allergic reactions, or similar reactions, and has a reasonable benefit/risk ratio.

The description of the range of values in the text is intended to be used as a shorthand for the individual value that falls into the range. Unless otherwise stated herein, each individual value is incorporated into this specification as if it were referenced separately herein.

The term “weight percentage” or “percentage in a weight per weight basis” or “wt %” is defined as the weight of the individual components in the composition divided by the total weight of all components of the composition and then multiplied by 100%. In some cases, if the composition has an outer coat, the weight of the outer coat may be comprised or excluded from the total weight.

### Examples

In order to enable those skilled in the art to better understand the technical solution of the present invention, some unrestricted embodiments are further disclosed below to further elaborate on the present invention.

The reagents used in the present invention can be purchased from the market or can be prepared by the method described in the present invention.

Unless otherwise indicated, the manufacturer of the reference listed drug (RLD) described in the examples and in the comparative examples is Xi′an Janssen, the specification is 60 mg, and the batch number is 19LG0585.

The term “specification” means the weight of the active ingredient in a single dose of the formulation, e.g., an apalutamide nanocrystalline tablet with a specification of 60 mg means that each apalutamide nanocrystalline tablet contains 60 mg of apalutamide.

### Comparative Example 1: Dissolution Test of Apalutamide Tablets Obtained without Milling

Composition prescription: as shown in Table 1.

TABLE 1
Apalutamide Tablets Prescription

| | Prescription number | |
|---|---|---|
| | Comparative prescription 1 | |
| Component | Single dose mg/tablet | Prescription ratio (%) |
| Apalutamide | 60.00 | 20.00 |
| Hydroxypropyl cellulose | 6.00 | 2.00 |
| Sodium dodecyl sulfate | 6.00 | 2.00 |
| Mannitol | 206.10 | 68.70 |
| Croscarmellose sodium | 15.00 | 5.00 |
| Micronized silica gel | 3.90 | 1.30 |
| Magnesium stearate | 3.00 | 1.00 |
| Total | 300 | 100.00 |

Preparation process: According to the prescription ratio in Table 1, 0.48 g of hydroxypropyl cellulose, 0.48 g of sodium dodecyl sulfate (SDS), 4.80 g of apalutamide pharmaceutical active ingredient (API), 16.49 g of mannitol, 1.20 g of croscarmellose sodium, 0.31 g of micronized silica gel and 0.24 g of magnesium stearate were weighed, mixed evenly and compressed, then apalutamide tablets were obtained.

Dissolution test: The apalutamide tablets obtained in comparative example 1 were tested in vitro dissolution by paddle method under the conditions of pH 6.8, volume of dissolution medium 900 mL, temperature of 37.0±0.5° C., and 75 rpm, and the sampling time points of dissolution were 5 min, 10 min, 15 min, 20 min, 30 min and 60 min. Sampling location: from the top of the paddle blade to the midpoint of the liquid level, 10 mm away from the inner wall of the dissolution cup. The high performance liquid chromatography method was used for the determination of dissolution content in vitro. The test results are shown in Table 2.

TABLE 2
Dissolution Results of Apalutamide
Tablets without Milling (%, n = 3)

| | 5 | 10 | 15 | 20 | 30 | 60 |
|---|---|---|---|---|---|---|
| Dissolution medium | min | min | min | min | min | min |
| pH 1.2 buffer | 6 | 7 | 9 | 9 | 10 | 11 |
| pH 4.5 buffer | 7 | 8 | 10 | 11 | 12 | 13 |
| pH 6.8 buffer | 7 | 8 | 10 | 11 | 12 | 15 |
| Buffer containing 0.25 | 23 | 32 | 37 | 40 | 44 | 50 |
| wt % SDS at pH 4.5 | | | | | | |

Result analysis: The dissolution rate of apalutamide tablets obtained without milling was small and the dissolution speed was slow.

### Example 1: Screening of Spatial Stabilizer Types

Composition prescription: as shown in Table 3.

TABLE 3
Prescription Table for Screening Types of Spatial Stabilizer

| | Single dose mg/tablet | | | | | | | | | | |
|---|---|---|---|---|---|---|---|---|---|---|---|
| | | | Pre- | Pre- | Pre- | Pre- | Pre- | Comparative | Comparative | Comparative | Comparative |
| | | | scrip- | scrip- | scrip- | scrip- | scrip- | prescription | prescription | prescription | prescription |
| Component | Prescription 1 | | tion 2 | tion 3 | tion 4 | tion 5 | tion 6 | 2 | 3 | 4 | 5 |
| Active | Apalutamide | 60 | 60 | 60 | 60 | 60 | 60 | 60 | 60 | 60 | 60 |
| ingredient | | | | | | | | | | | |
| Spatial | Povidone K17 | 30 | — | — | — | — | — | — | — | — | — |
| stabilizer | Hydroxypropyl | — | 30 | — | — | — | — | — | — | — | — |
| | cellulose | | | | | | | | | | |
| | Vinylpyrrolidone- | — | — | 30 | — | — | — | — | — | — | — |
| | vinyl acetate | | | | | | | | | | |
| | copolymer | | | | | | | | | | |
| | Polyvinyl alcohol | — | — | — | 30 | — | — | — | — | — | — |
| | Hydroxypropyl | — | — | — | — | 30 | — | — | — | — | — |
| | methylcellulose | | | | | | | | | | |
| | E3 | | | | | | | | | | |
| | Hydroxypropyl | — | — | — | — | — | 30 | — | — | — | — |
| | methylcellulose | | | | | | | | | | |
| | E5 | | | | | | | | | | |
| | Soluplus | — | — | — | — | — | — | 30 | — | — | — |
| | Tween-80 | — | — | — | — | — | — | — | 30 | — | — |
| | Poloxamer 188 | — | — | — | — | — | — | — | — | 30 | — |
| | Polyvinyl | — | — | — | — | — | — | — | — | — | 30 |
| | alcohol 6000 | | | | | | | | | | |
| Ionic | Sodium dodecyl | 6 | 6 | 6 | 6 | 6 | 6 | 6 | 6 | 6 | 6 |
| surfactants | sulfate | | | | | | | | | | |
| Protective | Mannitol | 78 | 78 | 78 | 78 | 78 | 78 | 78 | 78 | 78 | 78 |
| agent | | | | | | | | | | | |
| Additional | Mannitol | 104.1 | 104.1 | 104.1 | 104.1 | 104.1 | 104.1 | 104.1 | 104.1 | 104.1 | 104.1 |
| Additional | Croscarmellose | 15 | 15 | 15 | 15 | 15 | 15 | 15 | 15 | 15 | 15 |
| excipients | sodium | | | | | | | | | | |
| | Micronized | 3.9 | 3.9 | 3.90 | 3.9 | 3.9 | 3.9 | 3.9 | 3.9 | 3.9 | 3.9 |
| | silica gel | | | | | | | | | | |
| | Magnesium | 3 | 3 | 3 | 3 | 3 | 3 | 3 | 3 | 3 | 3 |
| | stearate | | | | | | | | | | |

| | | | | | | | | | | |
|---|---|---|---|---|---|---|---|---|---|---|
| Total | 300 | 300 | 300 | 300 | 300 | 300 | 300 | 300 | 300 | 300 |

### Preparation Process:

According to Table 3, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 2: Dissolution Test

The reference listed drug (the manufacturer is Xi′an Janssen, the specification is 60 mg, and the batch number is 19LG0585) and the apalutamide nanocrystalline tablets prepared by prescription 1˜6 and comparative prescription 2˜5 in example 1 were tested in vitro dissolution by paddle method under the conditions of pH 6.8, volume of dissolution medium 900 mL, temperature of 37.0±0.5° C., and 75 rpm, and the sampling time points of dissolution were 5 min, 10 min, 15 min, 20 min, 30 min and 60 min.

Sampling location: from the top of the paddle blade to the midpoint of the liquid level, 10 mm away from the inner wall of the dissolution cup. The high performance liquid chromatography method was used for the determination of dissolution content in vitro. The test results are shown in Table 4.

TABLE 4
Dissolution Results of Prescriptions 1~6, Comparative Prescriptions
2~5 and Reference Listed Drug (%, n = 3)

| | 5 | 10 | 15 | 20 | 30 | 60 |
|---|---|---|---|---|---|---|
| Prescription number | min | min | min | min | min | min |
| Prescription 1 | 40 | 72 | 89 | 94 | 94 | 93 |
| Prescription 2 | 42 | 76 | 92 | 92 | 93 | 94 |
| Prescription 3 | 30 | 56 | 75 | 85 | 88 | 87 |
| Prescription 4 | 43 | 57 | 88 | 92 | 94 | 94 |
| Prescription 5 | 35 | 66 | 78 | 86 | 87 | 87 |
| Prescription 6 | 33 | 61 | 73 | 81 | 84 | 86 |
| Comparative | 7 | 8 | 10 | 11 | 12 | 15 |
| prescription 1 | | | | | | |
| Comparative | 9 | 20 | 33 | 46 | 67 | 68 |
| prescription 2 | | | | | | |
| Comparative | 7 | 17 | 26 | 30 | 31 | 28 |
| prescription 3 | | | | | | |
| Comparative | 12 | 24 | 32 | 32 | 34 | 37 |
| prescription 4 | | | | | | |
| Comparative | 6 | 8 | 9 | 9 | 9 | 10 |
| prescription 5 | | | | | | |
| Reference listed | 59 | 72 | 74 | 81 | 81 | 87 |
| drug (19LG0585) | | | | | | |

### Result Analysis:

Under the same conditions, the apalutamide nanocrystalline tablets with prescriptions 1˜6 using povidone K17, hydroxypropyl cellulose, vinylpyrrolidone-vinyl acetate copolymer, polyvinyl alcohol, hydroxypropyl methylcellulose E5, hydroxypropyl methylcellulose E3 as spatial stabilizers were more higher dissolution rate and faster dissolution speed in pH 6.8 medium than the apalutamide nanocrystalline tablets with comparative prescription 2˜5 using Soluplus, Tween-80, Poloxamer P188 and polyethylene glycol 6000 as spatial stabilizers; so at least one of povidone K17, vinyl pyrrolidone-vinyl acetate copolymer, polyvinyl alcohol, hydroxypropyl methylcellulose E5 and hydroxypropyl methylcellulose E3 is preferred as spatial stabilizer.

### Example 3: Screening of the Proportion of Spatial Stabilizers

Composition prescriptions: as shown in Tables 5 and 6.

TABLE 5
Screening Prescription Table of Spatial Stabilizers Ratio

| | | Single dose mg/tablet | | | | | |
|---|---|---|---|---|---|---|---|
| | | Prescription | Prescription | Prescription | Prescription | Prescription | Prescription |
| Component | | 2 | 8 | 9 | 10 | 11 | 12 |
| Active ingredient | Apalutamide | 60 | 60 | 60 | 60 | 60 | 60 |
| Spatial stabilizer | Hydroxypropyl | 30 | 17.14 | 12 | 7.5 | 6 | 2 |
| | cellulose | | | | | | |
| Ionic surfactants | Sodium dodecyl | 6 | 6 | 6 | 6 | 6 | 6 |
| | sulfate | | | | | | |
| Protective agent | Mannitol | 78 | 78 | 78 | 78 | 78 | 78 |
| AdditionalAddi- | Mannitol | 104.10 | 116.96 | 122.10 | 126.60 | 128.10 | 132.1 |
| tional excipients | Croscarmellose | 15 | 15 | 15 | 15 | 15 | 15 |
| | sodium | | | | | | |
| | Micronized | 3.9 | 3.9 | 3.9 | 3.9 | 3.9 | 3.9 |
| | silica gel | | | | | | |
| | Magnesium | 3 | 3 | 3 | 3 | 3 | 3 |
| | stearate | | | | | | |

| | | | | | | |
|---|---|---|---|---|---|---|
| Total | 300 | 300 | 300 | 300 | 300 | 300 |

TABLE 6
Spatial Stabilizers Ratio Screening Prescription Table

| | | Single dose mg/tablet | | | | | | | |
|---|---|---|---|---|---|---|---|---|---|
| | | | | | | | | Comparative | Comparative |
| | | Prescrip- | Prescrip- | Prescrip- | Prescrip- | Prescrip- | Prescrip- | prescription | prescription |
| Component | | tion 13 | tion 1 | tion 14 | tion 15 | tion 16 | tion 17 | 6 | 7 |
| Active ingredient | Apalutamide | 60 | 60 | 60 | 60 | 60 | 60 | 60 | 60 |
| Spatial stabilizer | Povidone K17 | 120 | 30 | 17.14 | 12 | 6 | 4 | 150 | — |
| Ionic surfactants | Sodium dodecyl | 6 | 6 | 6 | 6 | 6 | 6 | 6 | 6 |
| | sulfate | | | | | | | | |
| Protective agent | Mannitol | 78 | 78 | 78 | 78 | 78 | 78 | 78 | 78 |
| AdditionalAddi- | Mannitol | 14.1 | 104.1 | 116.96 | 122.1 | 128.1 | 130.1 | 34.1 | 134.1 |
| tional excipients | Croscarmellose | 15 | 15 | 15 | 15 | 15 | 15 | 15 | 15 |
| | sodium | | | | | | | | |
| | Micronized | 3.9 | 3.9 | 3.9 | 3.9 | 3.9 | 3.9 | 3.9 | 3.9 |
| | silica gel | | | | | | | | |
| | Magnesium | 3 | 3 | 3 | 3 | 3 | 3 | 3 | 3 |
| | stearate | | | | | | | | |

| | | | | | | | | |
|---|---|---|---|---|---|---|---|---|
| Total | 300 | 300 | 300 | 300 | 300 | 300 | 350 | 300 |

### Preparation of Prescriptions 1, 2, 8-17 and Comparative Prescription 6:

According to Table 5 or Table 6, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Preparation of Comparative Prescription 7:

0.48 g of sodium dodecyl sulfate was weighed, water was added and stirred until the solution was clear, and then the prescribed amount of apalutamide was added to stir to obtain a suspension. Milling medium and suspension were added to the milling chamber for milling, no matter how long milling, apalutamide clumps together in the form of large particles and could not be mill evenly.

### Example 4: Dissolution Test

The apalutamide nanocrystalline tablets prepared in prescriptions 1, 2, 8-17 in example 3 and comparative prescriptions 6-7 were respectively taken for dissolution test, and the dissolution operation was the same as in example 2. The test results are shown in Table 7.

TABLE 7
Dissolution Results of Prescriptions 1, 2, 8~17 and
Comparative Prescriptions 6-7 (%, n = 3)

| | 5 | 10 | 15 | 20 | 30 | 60 |
|---|---|---|---|---|---|---|
| Prescription number | min | min | min | min | min | min |
| Prescription 1 | 40 | 72 | 89 | 94 | 94 | 93 |
| Prescription 2 | 42 | 76 | 92 | 92 | 93 | 94 |
| Prescription 8 | 47 | 80 | 90 | 91 | 90 | 90 |
| Prescription 9 | 61 | 90 | 90 | 90 | 94 | 89 |
| Prescription 10 | 70 | 88 | 89 | 90 | 91 | 88 |
| Prescription 11 | 42 | 76 | 92 | 92 | 93 | 94 |
| Prescription 12 | 45 | 76 | 87 | 93 | 93 | 93 |
| Prescription 13 | 14 | 37 | 60 | 69 | 91 | 89 |
| Prescription 14 | 19 | 50 | 72 | 86 | 91 | 88 |
| Prescription 15 | 33 | 74 | 88 | 93 | 98 | 101 |
| Prescription 16 | 37 | 71 | 84 | 88 | 93 | 97 |
| Prescription 17 | 51 | 65 | 77 | 87 | 91 | 92 |
| Comparative | 6 | 17 | 30 | 35 | 41 | 42 |
| prescription 6 | | | | | | |
| Comparative | 21 | 34 | 44 | 48 | 52 | 51 |
| prescription 7 | | | | | | |
| Reference listed | 59 | 72 | 74 | 81 | 81 | 87 |
| drug (19LG0585) | | | | | | |

Result analysis: Povidone K17 or hydroxypropyl cellulose was used as the spatial stabilizer, and the weight ratio of the spatial stabilizer to apalutamide was in the range of 1:30˜1:2, and the apalutamide nanocrystalline tablets (prescriptions 8-17) had high dissolution rate and fast dissolution speed in pH 6.8 medium.

In apalutamide nanocrystalline tablets, the selected spatial stabilizer mainly provides steric hindrance effect to achieve inter-particle stability in the system. Too much steric stabilizer will lead to an increase in the viscosity of the milling system, which is not conducive to the acquisition of nanoscale particles in a short time, and will even lead to a decrease in dissolution. And too little spatial stabilizer is not enough to form an ideal space steric effect between particles, particles between prone to coalescence, also not conducive to the physical stability of the final preparations. Therefore, the weight ratio of the spatial stabilizer to apalutamide in the range of 1:15 to 1:2 can improve the dissolution rate of apalutamide nanocrystal tablets.

### Example 5: Screening of the Proportion of Ionic Surfactant

Composition prescription: as shown in Table 8.

TABLE 8
Screening Prescription Table of Ionic Surfactant Ratio

| | | Single dose mg/tablet | | | | | | |
|---|---|---|---|---|---|---|---|---|
| | | | | | | | Comparative | Comparative |
| | | Prescription | Prescription | Prescription | Prescription | Prescription | prescription | prescription |
| Component | | 19 | 20 | 11 | 21 | 22 | 8 | 9 |
| Active | Apalutamide | 60 | 60 | 60 | 60 | 60 | 60 | 60 |
| ingredient | | | | | | | | |
| Spatial | Povidone K17 | 17.14 | 17.14 | 17.14 | 17.14 | 17.14 | 17.14 | 17.14 |
| stabilizer | | | | | | | | |
| Ionic | Sodium dodecyl | 30 | 12 | 6 | 3 | 0.03 | — | 60 |
| surfactants | sulfate | | | | | | | |
| Protective | Mannitol | 78 | 78 | 78 | 78 | 78 | 78 | 78 |
| agent | | | | | | | | |
| Additional | Mannitol | 92.96 | 110.96 | 116.96 | 119.96 | 122.93 | 122.96 | 62.96 |
| excipients | Croscarmellose | 15 | 15 | 15 | 15 | 15 | 15 | 15.00 |
| | sodium | | | | | | | |
| | Micronized | 3.9 | 3.9 | 3.9 | 3.9 | 3.9 | 3.9 | 3.90 |
| | silica gel | | | | | | | |
| | Magnesium | 3 | 3 | 3 | 3 | 3 | 3 | 3.00 |
| | stearate | | | | | | | |

| | | | | | | | |
|---|---|---|---|---|---|---|---|
| Total | 300 | 300 | 300 | 300 | 300 | 300 | 300 |

### Preparation Process:

According to Table 8, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

Comparative prescription 8 could not be mill to nanoscale particles.

Comparative prescription 9 produced a large number of bubbles during the milling process.

### Example 6: Dissolution Test

The apalutamide nanocrystalline tablets prepared in prescriptions 11, 19-22 in example 5 were respectively taken for dissolution test, and the dissolution operation was the same as in example 2. The test results are shown in Table 9.

TABLE 9
Dissolution Results of Prescriptions 11, 19~22 (%, n = 3)

| | 5 | 10 | 15 | 20 | 30 | 60 |
|---|---|---|---|---|---|---|
| Prescription number | min | min | min | min | min | min |
| Prescription 19 | 39 | 69 | 88 | 98 | 95 | 95 |
| Prescription 20 | 40 | 70 | 88 | 97 | 91 | 97 |
| Prescription 11 | 61 | 90 | 90 | 90 | 94 | 89 |
| Prescription 21 | 39 | 67 | 79 | 83 | 83 | 84 |
| Prescription 22 | 45 | 64 | 75 | 84 | 84 | 84 |
| Reference listed | 59 | 72 | 74 | 81 | 81 | 87 |
| drug (19LG0585) | | | | | | |

Result analysis: The above dissolution results showed that the apalutamide nanocrystalline tablets obtained in the pH 6.8 medium with a weight ratio of 1:2000-1:2 of sodium dodecyl sulfate as an ionic surfactant to apalutamide had high dissolution rate and fast dissolution speed. In the apalutamide nanocrystalline composition, the ionic surfactant mainly achieves the stability effect between particles in the system through the potential effect, and the ionic surfactant has solubilization effect on apalutamide. However, adding too much ionic surfactant will lead to a large number of bubbles in the milling process, which is not conducive to milling, and too much dosage is easy to cause toxicity problems, therefore, the preferred range of ionic surfactant is that the weight ratio of ionic surfactant to apalutamide is 1:2000-1:2.

### Example 7: Screening of the Rate of Protective Agent

Composition prescription: as shown in Table 10.

TABLE 10
Screening Prescription Table of Protective Agent Ratio

| | | | | | | | | | | Comparative |
|---|---|---|---|---|---|---|---|---|---|---|
| | | Prescrip- | Prescrip- | Prescrip- | Prescrip- | Prescrip- | Prescrip- | Prescrip- | Prescrip- | prescription |
| Component | | tion 24 | tion 11 | tion 25 | tion 26 | tion 27 | tion 17 | tion 28 | tion 29 | 10 |
| Active | Apalutamide | 60 | 60 | 60 | 60 | 60 | 60 | 60 | 60 | 60 |
| ingredient | | | | | | | | | | |
| Spatial | Hydroxypropyl | 6 | 6 | 6 | 6 | — | — | — | — | — |
| stabilizer | cellulose | | | | | | | | | |
| | Povidone K17 | — | — | — | — | 6 | 6 | 6 | 6 | 6 |
| Ionic | Sodium dodecyl | 6 | 6 | 6 | 6 | 6 | 6 | 6 | 6 | 6 |
| surfactants | sulfate | | | | | | | | | |
| Protective | Mannitol | 480 | 78 | 60 | 15 | 480 | 78 | 60 | 15 | — |
| agent | | | | | | | | | | |
| Additional | Mannitol | — | 128.1 | 146.1 | 191.1 | — | 128.1 | 146.1 | 191.1 | 206.1 |
| excipients | Croscarmellose | 30 | 15 | 15 | 15 | 30 | 15 | 15 | 15 | 15 |
| | sodium | | | | | | | | | |
| | Micronized | 8 | 3.9 | 3.9 | 3.9 | 8 | 3.9 | 3.9 | 3.9 | 3.9 |
| | silica gel | | | | | | | | | |
| | Magnesium | 6 | 3 | 3 | 3 | 6 | 3 | 3 | 3 | 3 |
| | stearate | | | | | | | | | |

| | | | | | | | | | |
|---|---|---|---|---|---|---|---|---|---|
| Total | 596 | 300 | 300 | 300 | 596 | 300 | 300 | 300 | 300 |

Preparation process: According to Table 10, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 8: Dissolution Test

The apalutamide nanocrystalline tablets prepared in prescriptions 11, 17, 24-29 in example 7 and comparative prescription 10 were respectively taken for dissolution test, and the dissolution operation was the same as in example 2. The test results are shown in Table 11.

TABLE 11
Dissolution Results of Prescriptions 11, 17, 24~29 and Comparative
Prescription 10 in pH 6.8 Medium (%, n = 3)

| | 5 | 10 | 15 | 20 | 30 | 60 |
|---|---|---|---|---|---|---|
| Prescription number | min | min | min | min | min | min |
| Prescription 24 | 61 | 89 | 88 | 90 | 94 | 102 |
| Prescription 11 | 37 | 71 | 84 | 88 | 93 | 97 |
| Prescription 25 | 26 | 68 | 94 | 96 | 100 | 97 |
| Prescription 26 | 29 | 68 | 87 | 91 | 94 | 98 |
| Prescription 27 | 58 | 88 | 90 | 91 | 95 | 98 |
| Prescription 17 | 70 | 88 | 89 | 90 | 91 | 88 |
| Prescription 28 | 51 | 89 | 94 | 92 | 91 | 92 |
| Prescription 29 | 52 | 92 | 92 | 95 | 93 | 93 |
| Comparative | 12 | 16 | 15 | 17 | 16 | 17 |
| prescription 10 | | | | | | |
| Reference listed | 59 | 72 | 74 | 81 | 81 | 87 |
| drug (19LG0585) | | | | | | |

Result analysis: The above dissolution results showed that the apalutamide nanocrystalline tablets with the weight ratio of the protective agent to apalutamide in the range of 8:1˜1:4 had high dissolution rate and fast dissolution speed in pH 6.8 medium.

In the apalutamide nanocrystal composition, it is important to add a suitable protective agent in order to reduce the damage during the solidification of the nanocrystals and maintain the redispersion property of the nanocrystals. Too much protective agent will lead to too much tablet weight; too little or no protectant protective agent, and the water will be lost instantaneously during the curing process, which will lead to the destruction of the nanocrystalline structure, easy to coalescence between particles, and poor redispersibility, which is not conducive to the physical stability of apalutamide nanocrystalline composition. In the present invention, the weight ratio of the protective agent to apalutamide is preferably 8:1˜1:4, which can not only achieve the technical effect of high dissolution rate and fast dissolution speed, but also reduce the weight of the tablet and improve the physical stability of the apalutamide nanocrystalline composition.

### Example 9: Screening of Curing Process—Spray Drying

Composition prescription: as shown in Table 12.

TABLE 12
Screening Prescription Table of Apalutamide
Nanocrystalline Tablets Curing Process

| | | Single dose mg/tablet | |
|---|---|---|---|
| | | Prescrip- | Prescrip- |
| Component | | tion 28 | tion 25 |
| Active ingredient | Apalutamide | 60 | 60 |
| Spatial stabilizer | Povidone K17 | 6 | 0 |
| | Hydroxypropyl cellulose | — | 6 |
| Ionic surfactant | Sodium dodecyl sulfate | 6 | 6 |
| Protective agent | Mannitol | 60 | 60 |
| Additional excipients | Mannitol | 146.1 | 146.1 |
| | Croscarmellose sodium | 15 | 15 |
| | Micronized silica gel | 3.9 | 3.9 |
| | Magnesium stearate | 3 | 3 |

| | | |
|---|---|---|
| Total | 300 | 300 |

### Preparation Process:

According to Table 12, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried and obtain a dry powder; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 10: Screening of Curing Process in Apalutamide Nanocrystalline Tablets-Freeze Drying

Composition prescription: as shown in Table 13.

TABLE 13
Curing Process in Apalutamide Nanocrystalline
Tablets Screening Prescription Table

| | | Single dose mg/tablet | |
|---|---|---|---|
| | | Prescrip- | Prescrip- |
| Component | | tion 31 | tion 32 |
| Active ingredient | Apalutamide | 60 | 60 |
| Spatial stabilizer | Povidone K17 | 6 | — |
| | Hydroxypropyl cellulose | — | 6 |
| Ionic surfactant | Sodium dodecyl sulfate | 6 | 6 |
| Protective agent | Mannitol | 60 | 60 |
| Additional excipients | Mannitol | 146.1 | 146.1 |
| | Croscarmellose sodium | 15 | 15 |
| | Micronized silica gel | 3.9 | 3.9 |
| | Magnesium stearate | 3 | 3 |

| | | |
|---|---|---|
| Total | 300 | 300 |

### Preparation Process:

According to Table 13, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent and freeze-dried to obtain the lyophilized substance. After milling the lyophilized material through a 30-mesh screen, the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 11: Screening of Curing Process in Apalutamide Nanocrystalline Tablets-Fluidized Bed Drying

Composition prescription: as shown in Table 14.

TABLE 14
Curing Process in Apalutamide Nanocrystalline Tablets Screening Prescription Table

| | | | Single dose mg/tablet | | | | |
|---|---|---|---|---|---|---|---|
| | | | Prescription | Prescription | Prescription | Prescription | Prescription |
| Component | | | 33 | 34 | 35 | 36 | 37 |
| Active ingredient | | Apalutamide | 60 | 60 | 60 | 60 | 60 |
| Spatial stabilizer | | Povidone K17 | 6 | — | — | — | — |
| | | Hydroxypropyl | — | 6 | 6 | 6 | 6 |
| | | cellulose | | | | | |
| Ionic surfactant | | Sodium dodecyl | 6 | 6 | 6 | 6 | 6 |
| | | sulfate | | | | | |
| Fluidized | Protective | Mannitol | 120 | 120 | — | — | — |
| bed spray | agent | Microcrystalline | — | — | 120 | — | — |
| drying | | cellulose | | | | | |
| | | Anhydrous calcium | — | — | — | 120 | — |
| | | hydrogen phosphate | | | | | |
| | | Microcrystalline | — | — | — | — | 198 |
| | | cellulose pellet core | | | | | |
| | N/A | Micronized silica gel | 1.2 | 1.2 | 1.2 | 1.2 | — |

| Additional | Mannitol | 88.8 | 88.8 | 88.8 | 88.8 | — |
|---|---|---|---|---|---|---|
| excipients | Croscarmellose | 15 | 15 | 15 | 15 | — |
| | sodium | | | | | |
| | Magnesium stearate | 3 | 3 | 3 | 3 | — |

| | | | | | |
|---|---|---|---|---|---|
| Total | 300 | 300 | 300 | 300 | 270 |

### Preparation Process:

According to Table 14, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; Using the fluidized bed spray drying process, the nano-suspension was sprayed on the substrate with the protective agent and/or micropowder silica gel as the substrate to obtain dry particles/drug-coated pellets. the prescribed amount of additional excipients mannitol, croscarmellose sodium and micronized silica gel were added and the mixture was mixed, and then magnesium stearate was added to dry particles, mixed and compressed, then apalutamide nanocrystalline tablets were obtained. The drug-coated pellets were poured into the capsule to obtain apalutamide nanocrystalline capsules.

### Example 12: Dissolution Test

The apalutamide nanocrystalline tablets prepared in prescriptions 25, 28 in example 9, prescriptions 32-32 in example 10, prescriptions 33-37 in example 11 were respectively taken for dissolution test, and the dissolution operation was the same as in example 2. The test results are shown in Table 15.

TABLE 15
Dissolution results of prescriptions 25, 28, 31~37 (%, n = 3)

| | 5 | 10 | 15 | 20 | 30 | 60 |
|---|---|---|---|---|---|---|
| Prescription number | min | min | min | min | min | min |
| Prescription 28 | 51 | 89 | 94 | 92 | 91 | 92 |
| Prescription 25 | 26 | 68 | 94 | 96 | 100 | 97 |
| Prescription 31 | 72 | 89 | 94 | 92 | 91 | 92 |
| Prescription 32 | 31 | 62 | 84 | 88 | 92 | 92 |
| Prescription 33 | 65 | 78 | 87 | 89 | 93 | 92 |
| Prescription 34 | 59 | 75 | 85 | 92 | 93 | 93 |
| Prescription 35 | 43 | 68 | 83 | 87 | 89 | 89 |
| Prescription 36 | 15 | 72 | 87 | 93 | 93 | 94 |
| Prescription 37 | 21 | 56 | 81 | 89 | 89 | 91 |
| Reference listed | 59 | 72 | 74 | 81 | 81 | 87 |
| drug (19LG0585) | | | | | | |

Result analysis: the above dissolution results show that using the prescription of the present invention, the curing process adopts spray drying, fluidized bed spray drying and freeze drying, and the apalutamide preparation with fast dissolution speed can be obtained.

In the curing process by fluidized bed spray drying, mannitol, microcrystalline cellulose, microcrystalline cellulose pellet core, and anhydrous calcium hydrogen phosphate were used as protective agents, and the dissolution is relatively fast.

### Example 13: Apalutamide Nanocrystalline Tablets of Different Specifications

Composition prescription: as shown in Table 16.

TABLE 16
Prescription of Apalutamide Nanocrystalline Tablets of Different Specifications

| | | Specification | | |
|---|---|---|---|---|
| | | prescription 39-1 | prescription 39-2 | prescription 39-3 |
| Component | | 60 mg/tablet | 120 mg/tablet | 240 mg/tablet |
| Active ingredient | Apalutamide | 60 | 120 | 240 |
| Spatial stabilizer | Povidone K17 | 6 | 12 | 24 |
| Ionic surfactants | Sodium dodecyl sulfate | 6 | 12 | 24 |
| Protective agent | Mannitol | 60 | 120 | 240 |
| Additional | Mannitol | 99.75 | 199.5 | 399 |
| excipients | Croscarmellose sodium | 12.5 | 25 | 50 |
| | Micronized silica gel | 3.25 | 6.5 | 13 |
| | Magnesium stearate | 2.5 | 5 | 10 |

| | | | |
|---|---|---|---|
| Total | 250 | 500 | 1000 |
| Drug loading (%) | 24 | 24 | 24 |

### Preparation Process:

According to the prescription described in Table 16, apalutamide nanocrystalline tablets were prepared according to the preparation process described in example 9, and the specifications of apalutamide in the tablets were 60 mg/tablet, 120 mg/tablet, and 240 mg/tablet, respectively.

### Example 14: Apalutamide Nanocrystalline Tablets of Different Specifications

Composition prescription: as shown in Table 17.

TABLE 17
Prescription of Apalutamide Nanocrystalline Tablets of Different Specifications

| | | Specification | | |
|---|---|---|---|---|
| | | prescription 40-1 | prescription 40-2 | prescription 40-3 |
| Component | | 60 mg/tablet | 120 mg/tablet | 240 mg/tablet |
| Active ingredient | Apalutamide | 60 | 120 | 240 |
| Spatial stabilizer | Hydroxypropyl cellulose | 6 | 12 | 24 |
| Ionic surfactant | Sodium dodecyl sulfate | 6 | 12 | 24 |
| Protective agent | Mannitol | 60 | 120 | 240 |
| Additional | Mannitol | 99.75 | 199.5 | 399 |
| excipients | Croscarmellose sodium | 12.5 | 25 | 50 |
| | Micronized silica gel | 3.25 | 6.5 | 13 |
| | Magnesium stearate | 2.5 | 5 | 10 |

| | | | |
|---|---|---|---|
| Total | 250 | 500 | 1000 |
| Drug loading (%) | 24 | 24 | 24 |

### Preparation Process:

According to the prescription described in Table 17, apalutamide nanocrystalline tablets were prepared according to the preparation process described in example 7, and the specifications of apalutamide in the tablets were 60 mg/tablet, 120 mg/tablet, and 240 mg/tablet, respectively.

### Example 15: Dissolution Results

The apalutamide nanocrystalline tablets prepared in example 13 and 14 were respectively taken for dissolution test, and the dissolution operation was the same as in example 2. The test results are shown in Table 18.

TABLE 18
Dissolution Results of Apalutamide Nanocrystalline
Tablets of Different Specifications (%, n = 3)

| Dissolution | Prescription | | | 5 | 10 | 15 | 20 | 30 | 60 |
|---|---|---|---|---|---|---|---|---|---|
| medium | number | Specification | | min | min | min | min | min | min |
| pH 6.8 | Prescription | 60 | mg | 32 | 67 | 88 | 94 | 95 | 95 |
| buffer | 39-1 | | | | | | | | |
| | Prescription | 120 | mg | 36 | 67 | 86 | 96 | 99 | 98 |
| | 39-2 | | | | | | | | |
| | Prescription | 240 | mg | 44 | 75 | 92 | 97 | 97 | 96 |
| | 39-3 | | | | | | | | |
| | Prescription | 60 | mg | 22 | 58 | 85 | 93 | 102 | 100 |
| | 40-1 | | | | | | | | |
| | Prescription | 120 | mg | 29 | 66 | 98 | 103 | 99 | 105 |
| | 40-2 | | | | | | | | |
| | Prescription | 240 | mg | 32 | 62 | 87 | 100 | 102 | 103 |
| | 40-3 | | | | | | | | |

Result analysis: Apalutamide nanocrystalline tablets of different apalutamide specifications can be quickly dissolved.

### Example 16: Apalutamide Nanocrystalline Tablets of Different Tablet Weight

Composition prescription: as shown in Table 19.

TABLE 19
Prescription of Apalutamide Nanocrystalline Tablets of Different Tablet Weight

| | | Tablet weight | | | |
|---|---|---|---|---|---|
| | | Prescription | Prescription | Prescription | Prescription |
| | | 42-1 | 42-2 | 42-3 | 42-4 |
| Component | | 500 mg/tablet | 400 mg/tablet | 200 mg/tablet | 150 mg/tablet |
| Active ingredient | Apalutamide | 60 | 60 | 60 | 60 |
| Spatial stabilizer | Povidone K17 | 6 | 6 | 6 | 6 |
| Ionic surfactant | Sodium dodecyl | 6 | 6 | 6 | 6 |
| | sulfate | | | | |
| Protective agent | Mannitol | 60 | 60 | 60 | 60 |
| Additional | Mannitol | 331.5 | 238.8 | 53.4 | 7.05 |
| excipients | Croscarmellose | 25 | 20 | 10 | 7.5 |
| | sodium | | | | |
| | Micronized silica gel | 6.5 | 5.2 | 2.6 | 1.95 |
| | Magnesium stearate | 5 | 4 | 2 | 1.5 |

| | | | | |
|---|---|---|---|---|
| Total | 500 | 400 | 200 | 150 |
| Drug loading (%) | 12 | 15 | 30 | 40 |

### Preparation Process:

According to Table 19, the prescribed amount of spatial stabilizer and sodium dodecyl sulfate were weighed, water was added and stirred until the solution was clear, and then the prescribed amount of apalutamide was added to stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained, the tablet weight of apalutamide nanocrystalline tablets was 500 mg/tablet, 400 mg/tablet, 200 mg/tablet, and 150 mg/tablet, respectively.

### Example 17: Apalutamide Nanocrystalline Tablets of Different Tablet Weight

Composition prescription: as shown in Table 20.

TABLE 20
Prescription of Apalutamide Nanocrystalline Tablets of Different Tablet Weight

| | | Tablet weight | | | |
|---|---|---|---|---|---|
| | | Prescription | Prescription | Prescription | Prescription |
| | | 43-1 | 43-2 | 43-3 | 43-4 |
| Component | | 500 mg/tablet | 400 mg/tablet | 200 mg/tablet | 150 mg/tablet |
| Active ingredient | Apalutamide | 60 | 60 | 60 | 60 |
| Spatial stabilizer | Hydroxypropyl | 6 | 6 | 6 | 6 |
| | cellulose | | | | |
| Ionic surfactant | Sodium dodecyl | 6 | 6 | 6 | 6 |
| | sulfate | | | | |
| Protective agent | Mannitol | 60 | 60 | 60 | 60 |
| Additional | Mannitol | 331.5 | 238.8 | 53.4 | 7.05 |
| excipients | Croscarmellose | 25 | 20 | 10 | 7.5 |
| | sodium | | | | |
| | Micronized silica gel | 6.5 | 5.2 | 2.6 | 1.95 |
| | Magnesium stearate | 5 | 4 | 2 | 1.5 |

| | | | | |
|---|---|---|---|---|
| Total | 500 | 400 | 200 | 150 |
| Drug loading (%) | 12 | 15 | 30 | 40 |

### Preparation Process

According to Table 20, the prescribed amount of spatial stabilizer and sodium dodecyl sulfate were weighed, water was added and stirred until the solution was clear, and then the prescribed amount of apalutamide was added and the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixtire was mixed, and then magnesium stearate was added, mixed and compressed, then apalutamide nanocrystalline tablets were obtained, the tablet weight of apalutamide nanocrystalline tablets was 500 mg/tablet, 400 mg/tablet, 200 mg/tablet, and 150 mg/tablet, respectively.

### Example 18: Dissolution Test

The apalutamide nanocrystalline tablets prepared in example 16 and 17 were respectively taken for dissolution test, and the dissolution operation was the same as in example 2. The test results are shown in Table 21.

TABLE 21
Dissolution Results of Apalutamide Nanocrystalline
Tablets of Different Tablet Weight (%, n = 3)

| | Prescription | Tablet | 5 | 10 | 15 | 20 | 30 | 60 |
|---|---|---|---|---|---|---|---|---|
| Specification | number | weight | min | min | min | min | min | min |
| 60 mg | Prescription | 500 mg | 76 | 91 | 92 | 92 | 93 | 98 |
| | 42-1 | | | | | | | |
| | Prescription | 400 mg | 72 | 90 | 92 | 94 | 93 | 95 |
| | 42-2 | | | | | | | |
| | Prescription | 200 mg | 34 | 75 | 92 | 97 | 97 | 96 |
| | 42-3 | | | | | | | |
| | Prescription | 150 mg | 29 | 58 | 86 | 93 | 102 | 100 |
| | 42-4 | | | | | | | |
| 60 mg | Prescription | 500 mg | 61 | 84 | 88 | 94 | 94 | 104 |
| | 43-1 | | | | | | | |
| | Prescription | 400 mg | 64 | 86 | 89 | 97 | 97 | 97 |
| | 43-2 | | | | | | | |
| | Prescription | 200 mg | 33 | 67 | 91 | 100 | 101 | 96 |
| | 43-3 | | | | | | | |
| | Prescription | 150 mg | 43 | 58 | 85 | 93 | 102 | 100 |
| | 43-4 | | | | | | | |

Result analysis: Apalutamide nanocrystalline tablets with a tablet weight of 150 mg-500 mg can be quickly dissolved.

### Example 19: Investigation of Different Dosage Forms—Apalutamide Nanocrystalline Tablets

Composition prescription: as shown in Table 22.

TABLE 22
Apalutamide Nanocrystalline Tablet Prescriptions

| | | Single dose mg/tablet | |
|---|---|---|---|
| | | Prescription | Prescription |
| Component | | 46 | 47 |
| Active ingredient | Apalutamide | 60 | 60 |
| Spatial stabilizer | Povidone K17 | 6 | — |
| | Hydroxypropyl cellulose | — | 6 |
| Ionic surfactant | Sodium dodecyl sulfate | 6 | 6 |
| Protective agent | Mannitol | 60 | 60 |
| Additional | Mannitol | 99.75 | 99.75 |
| excipients | Croscarmellose sodium | 12.5 | 12.5 |
| | Micronized silica gel | 3.25 | 3.25 |
| | Magnesium stearate | 2.5 | 2.5 |

| | | |
|---|---|---|
| Total | 250 | 250 |

### Preparation Process:

According to Table 22, the prescribed amount of spatial stabilizer and Ionic surfactant were weighed, water was added and stirred until the solution was clear, and then the apalutamide was added and mixed to obtain a suspension; milling medium and suspension were added to the milling chamber for milling to obtain a nanocrystalline suspension; the nano suspension was mixed with the protective agent, spray dried; the additional excipients mannitol, croscarmellose sodium and micronized silica gel were added and the mixture was mixed, and then magnesium stearate was added, mixed to obtain total mixed particles, then apalutamide nanocrystalline tablets were obtained by compressing.

### Example 20: Investigation of Different Dosage Forms of Apalutamide Nanocrystalline Capsules

Prescription 48: The total mixed particles of prescription 46 were filled into capsules to obtain apalutamide nanocrystalline capsules;

Prescription 49: The total mixed particles of prescription 47 were filled into capsules to obtain apalutamide nanocrystalline capsules.

### Example 21: Investigation of Different Dosage Forms of Apalutamide Nanocrystalline Suspension

Preparation process of prescription 50: 0.48 g of hydroxypropyl cellulose and 0.48 g of sodium dodecyl sulfate were weighed, water was added and the mixture was stirred until the solution was clear, and then 4.80 g of apalutamide was added and mixed to obtain a suspension. Milling medium and suspension were added to the milling chamber for milling to obtain a apalutamide nanocrystalline suspension; and the nanocrystalline oral suspension was obtained after filtration.

### Example 22: Investigation of Different Dosage Forms of Apalutamide Nanocrystalline Suspension

Preparation process of prescription 51: 0.48 g of povidone K17 and 0.48 g of sodium dodecyl sulfate were weighed, water was added and the mixture was stirred until the solution was clear, and then 4.80 g of apalutamide was added and mixed to obtain a suspension. Milling medium and suspension were added to the milling chamber for milling to obtain a apalutamide nanocrystalline suspension; and the nanocrystalline oral suspension was obtained after filtration.

### Example 23: Dissolution Data

The apalutamide nanocrystalline tablets, apalutamide nanocrystalline capsules or apalutamide nanocrystalline suspension prepared in prescriptions 46-51 in examples 19-22 were respectively taken for dissolution test, and the dissolution operation was the same as in example 2. The test results are shown in Table 23.

TABLE 23
Dissolution Results of Prescriptions 46~51 (%, n = 3)

| | 5 | 10 | 15 | 20 | 30 | 60 |
|---|---|---|---|---|---|---|
| Prescription number | min | min | min | min | min | min |
| Prescription 46 | 43 | 79 | 92 | 92 | 93 | 98 |
| Prescription 47 | 42 | 76 | 92 | 94 | 93 | 95 |
| Prescription 48 | 23 | 58 | 88 | 92 | 93 | 93 |
| Prescription 49 | 16 | 59 | 86 | 94 | 97 | 103 |
| Prescription 50 | 94 | 95 | 95 | 97 | 95 | 97 |
| Prescription 51 | 98 | 99 | 99 | 100 | 100 | 100 |

Result analysis: The apalutamide nanocrystalline tablets, apalutamide nanocrystalline capsules and apalutamide nanocrystalline suspension were all rapidly dissolved in the dissolution medium at pH 6.8, and the dissolution speed of apalutamide nanocrystalline suspension was the fastest, followed by apalutamide nanocrystalline tablets.

### Example 24: Particle Size Stability

The apalutamide nanocrystalline oral suspensions prepared by prescription 50 in example 21 and prescription 51 in example 22 were taken respectively, and the particle size distribution of apalutamide nanocrythms oral suspension placed at room temperature for 0 day and 14 days after milling were detected by laser particle size analyzer, and the test results are shown in Table 24.

TABLE 24
Particle Size Stability Result

| | Particle size (μm) | Particle size (μm) at room |
|---|---|---|
| | for 0 day | temperature for 14 days |
| Prescription number | D90 | D90 |
| Prescription 50 | 0.463 | 0.441 |
| Prescription 51 | 0.452 | 0.545 |

Result analysis: The particle size of the nanocrystalline suspension is stable at room temperature.

### Example 25: Screening of Povidone Molecular Weight

Preparation process of prescription 54: 0.96 g of povidones of different molecular weights and 0.24 g of sodium dodecyl sulfate were weighed, water was added and the mixture was stirred until the solution was clear, and then 4.8 g of apalutamide was added and mixed to obtain a suspension; milling medium and suspension were added to the milling chamber for milling, and the apalutamide nanocrystalline suspension was obtained after filtration. The nanocrystalline suspension was mixed with 6.24 g mannitol and spray-dried to obtain a dry powder; additional excipients 13.72 g of glycine, 1.4 g of croscarmellose sodium and 0.36 g of micronized silica gel were added and the mixture was mixed, and then 0.28 g of magnesium stearate was added, mixed and compressed, then apalutamide nanocrystalline tablets were obtained.

Detection: The particle size distribution of apalutamide nanocrythms suspension placed at room temperature for 0 h and 96 h after milling, and the particle size distribution of the cured spray powder after redissolving in purified water were detected by laser particle size analyzer. The particle size distribution detection results are shown in Table 25. In addition, the finished apalutamide nanocrystalline tablets were taken for dissolution test, and the dissolution operation was the same as in example 2. The test results are shown in Table 26.

TABLE 25
Particle Size Stability of Apalutamide Nanocrystalline Suspensions Obtained by Povidone
by Different Molecular Weights and Particle Aize after Curing and Reconstitution

| | | Nanocrystalline suspension | | |
|---|---|---|---|---|
| | | D90 | | |
| Povidone | Povidone average | Particle size after | Particle size placed | Particle size after curing |
| models | molecular weight | milling (μm) | for 96 h (μm) | and reconstitution (μm) |
| K12 | About 2500 | 0.571 | 0.673 | 0.624 |
| K17 | About 10000 | 0.447 | 0.619 | 0.573 |
| K30 | About 50000 | 0.457 | 0.646 | 0.664 |
| K90 | About 1000000 | 2.204 | 2.359 | — |

Result analysis: When the molecular weight of povidone is large, the drug nanocrystals can not be obtained by milling, which may have high viscosity and low milling efficiency, and is not conducive to the dispersion of nanoparticles, and povidone with models of K12, K17 and K30, that is, the average molecular weight of ≤50000 can be used as a spatial stabilizer to obtain nanocrystals with stable particle size.

TABLE 26
Dissolution Results of Apalutamide Nanocrystalline
Tablets from Prescription 54 (%, n = 3)

| Prescription | | Povidone | 5 | 10 | 15 | 20 | 30 | 60 |
|---|---|---|---|---|---|---|---|---|
| number | Specification | models | min | min | min | min | min | min |
| Prescription | 60 mg | K12 | 35 | 54 | 81 | 86 | 87 | 90 |
| 54 | | K17 | 69 | 78 | 87 | 94 | 93 | 94 |
| | | K30 | 25 | 51 | 82 | 84 | 84 | 84 |

Result analysis: Povidone with models of K12, K17 and K30, that is, povidone with an average molecular weight of ≤50,000 can be dissolved quickly as a spatial stabilizer.

### Example 26: Screening of Ionic Surfactant Species

Composition prescription: as shown in Table 27.

TABLE 27
Screening Prescription Table of Ionic Surfactant Species

| | | Single dose mg/tablet | | | | |
|---|---|---|---|---|---|---|
| | | | | | Comparative | Comparative |
| | | Prescription | Prescription | Prescription | prescription | prescription |
| Component | | 11 | 55 | 56 | 11 | 12 |
| Active | Apalutamide | 60 | 60 | 60 | 60 | 60 |
| ingredient | | | | | | |
| Spatial | Hydroxypropyl | 6 | 6 | 6 | 6 | 6 |
| stabilizer | cellulose | | | | | |
| Ionic | Sodium dodecyl | 6 | — | — | — | — |
| surfactants | sulfate | | | | | |
| | Docusate sodium | — | 6 | — | | |
| | Lecithin | — | — | 6 | | |
| | Octadecylamine | — | — | — | 6 | — |
| | (stearic acid) | | | | | |
| | hydrochloride | | | | | |
| | Stearic acid | — | — | — | | 6 |
| Protective | Mannitol | 78 | 78 | 78 | 78 | 78 |
| agent | | | | | | |
| Additional | Mannitol | 128.1 | 128.1 | 128.1 | 128.1 | 128.1 |
| excipients | Croscarmellose | 15 | 15 | 15 | 15 | 15 |
| | sodium | | | | | |
| | Micronized | 3.9 | 3.9 | 3.9 | 3.9 | 3.9 |
| | silica gel | | | | | |
| | Magnesium | 3 | 3 | 3 | 3 | 3 |
| | stearate | | | | | |

| | | | | | |
|---|---|---|---|---|---|
| Total | 300 | 300 | 300 | 300 | 300 |

### Preparation Process:

According to Table 27, the prescribed amount of hydroxypropyl cellulose and ionic surfactants were weighed, water was added and the mixture was stirred until the solution was clear, and then the prescribed amount of apalutamide was added the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

Comparative prescription 11 and comparative prescription 12 suspensions do not yield nanoscale particles after milling.

### Example 27: Dissolution Test

The apalutamide nanocrystalline tablets prepared by prescription 11, prescription 55 and prescription 56 in example 26 were respectively taken for dissolution test, and the dissolution operation was the same as in example 2. The test results are shown in Table 28.

TABLE 28
Dissolution Results of Prescription 11, Prescription
55 and Prescription 56 (%, n = 3)

| | 5 | 10 | 15 | 20 | 30 | 60 |
|---|---|---|---|---|---|---|
| Prescription number | min | min | min | min | min | min |
| Prescription 11 | 42 | 76 | 92 | 92 | 93 | 94 |
| Prescription 55 | 31 | 56 | 72 | 87 | 87 | 86 |
| Prescription 56 | 53 | 74 | 81 | 88 | 89 | 89 |
| Reference listed | 59 | 72 | 74 | 81 | 81 | 87 |
| drug (19LG0585) | | | | | | |

Result analysis: Sodium dodecyl sulfate, sodium docusate and lecithin were used as ionic surfactants, and the dissolution was fast.

### Example 28: Screening of Protective Agent Types

Composition prescription: as shown in Table 29.

TABLE 29
Screening Prescription Table of Protective Agent Types

| | | Single dose mg/tablet | | | | |
|---|---|---|---|---|---|---|
| | | Prescription | Prescription | Prescription | Comparative | Comparative |
| Component | | 11 | 58 | 59 | prescription 13 | prescription 14 |
| Active | Apalutamide | 60 | 60 | 60 | 60 | 60 |
| ingredient | | | | | | |
| Spatial | Hydroxypropyl | 6 | 6 | 6 | 6 | 6 |
| stabilizer | cellulose | | | | | |
| Ionic | Sodium dodecyl | 6 | 6 | 6 | 6 | 6 |
| surfactant | sulfate | | | | | |
| Protective | Mannitol | 78 | — | — | — | — |
| agent | Lactose | — | 78 | — | — | — |
| | Maltitol | — | — | 78 | — | — |
| | Sorbitol | — | — | — | 78 | — |
| | Cane sugar | — | — | — | — | 78 |
| Additional | Mannitol | 128.1 | 128.1 | 128.1 | 128.1 | 128.1 |
| excipients | Croscarmellose | 15 | 15 | 15 | 15 | 15 |
| | sodium | | | | | |
| | Micronized | 3.9 | 3.9 | 3.9 | 3.9 | 3.9 |
| | silica gel | | | | | |
| | Magnesium | 3 | 3 | 3 | 3 | 3 |
| | stearate | | | | | |

| | | | | | |
|---|---|---|---|---|---|
| Total | 300 | 300 | 300 | 300 | 300 |

Preparation process: According to Table 29, the prescribed amount of hydroxypropyl cellulose and Sodium dodecyl sulfate were weighed, water was added and the mixture was stirred until the solution was clear, and then the prescribed amount of apalutamide was added the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

When the nanocrystalline suspensions of comparative prescription 13 and the comparative prescription 14 were spray dried, the sprayed dry material became sticky and could not be further processed.

### Example 29: Dissolution Test

The apalutamide nanocrystalline tablets prepared by prescription 11, prescription 58 and prescription 59 in example 28 were respectively taken for dissolution test, and the dissolution operation was the same as in example 2. The test results are shown in Table 30.

TABLE 30
Dissolution Results of Prescription 11, Prescription 58 and
Prescription 59 in pH 6.8 Medium in Example 28
(%, n = 3)

| Prescription | | | | | | |
|---|---|---|---|---|---|---|
| number | 5 min | 10 min | 15 min | 20 min | 30 min | 60 min |
| Prescription 11 | 37 | 71 | 84 | 88 | 93 | 97 |
| Prescription 58 | 41 | 68 | 87 | 88 | 88 | 89 |
| Prescription 59 | 32 | 57 | 78 | 84 | 86 | 86 |
| Reference | 59 | 72 | 74 | 81 | 81 | 87 |
| listed drug | | | | | | |
| (19LG0585) | | | | | | |

Results: Mannitol, lactose and maltitol were used as protective agents in the spray drying process, and the dissolution was relatively fast.

### Comparative Example 2: Dissolution Test of Apalutamide Tablets Using Mesoporous Silicon as Solubilizer

Composition prescription: as shown in Table 31.

TABLE 31
Prescription of Using Mesoporous Silicon as a Solubilizer
Comparative prescription 15

| | Single dose |
|---|---|
| Component | mg/tablet |
| Apalutamide | 60 |
| Mesoporous silicon | 140 |
| Microcrystalline cellulose | 78.1 |
| Croscarmellose sodium | 15 |
| Micronized silica gel | 3.9 |
| Magnesium stearate | 3 |
| Total | 300 |

Preparation process: According to the prescription table shown in Table 31, mesoporous silicon and apalutamide were weighed, apalutamide was dissolved in acetonitrile, and then dropwise added to mesoporous silicon, the mixture was stirred while adding, and then dried to obtain drug-loaded mesoporous silicon, then filler, disintegrant and lubricant was added and compressed.

Dissolution test: The tablets prepared in comparative prescription 15 were taken for dissolution test, and the dissolution operation was the same as in example 2. The test results are shown in Table 32.

TABLE 32
Dissolution Results of Apalutamide Tablets
Using Mesoporous Silicon as Solubilizer
(%, n = 3)

| Prescription | | | | | | |
|---|---|---|---|---|---|---|
| number | 5 min | 10 min | 15 min | 20 min | 30 min | 60 min |
| Comparative | 18 | 24 | 29 | 30 | 37 | 41 |
| prescription 15 | | | | | | |

Result analysis: The dissolution of tablets obtained by using mesoporous silicon as a solubilizer was slow.

### Example 30: Particle Size Distribution Before and After Curing

The apalutamide oral nanocrystalline tablets prepared from prescriptions 1-6, 8-17, 19-22, 24-29, 31-34, 55-56, 58-59 and comparison prescriptions 2-12 in the above examples were taken and the particle size distribution of the nanocrystalline suspension after milling before curing and reconstitution in purified water after curing was determined by a laser particle size analyzer, and the test results are shown in Table 33.

TABLE 33
Particle Size Distribution Before and After Curing

| | Particle size before | Particle size after |
|---|---|---|
| | curing (μm) | curing (μm) |
| Prescription number | D90 | D90 |
| Prescription 1 | 0.463 | 0.441 |
| Prescription 2 | 0.495 | 0.500 |
| Prescription 3 | 0.452 | 0.545 |
| Prescription 4 | 0.489 | 0.613 |
| Prescription 5 | 0.475 | 0.567 |
| Prescription 6 | 0.475 | 0.623 |
| Prescription 8 | 0.468 | 0.460 |
| Prescription 9 | 0.520 | 0.532 |
| Prescription 10 | 0.527 | 0.575 |
| Prescription 11 | 0.478 | 0.484 |
| Prescription 12 | 0.483 | 0.623 |
| Prescription 13 | 0.470 | 0.574 |
| Prescription 14 | 0.543 | 0.561 |
| Prescription 15 | 0.469 | 0.587 |
| Prescription 16 | 0.496 | 0.529 |
| Prescription 17 | 0.500 | 0.509 |
| Prescription 19 | 0.520 | 0.532 |
| Prescription 20 | 0.527 | 0.575 |
| Prescription 21 | 0.568 | 0.578 |
| Prescription 22 | 0.564 | 0.603 |
| Prescription 25 | 0.456 | 0.513 |
| Prescription 26 | 0.506 | 0.519 |
| Prescription 27 | 0.523 | 0.571 |
| Prescription 28 | 0.500 | 0.513 |
| Prescription 29 | 0.494 | 0.520 |
| Prescription 31 | 0.505 | 0.514 |
| Prescription 32 | 0.521 | 0.519 |
| Prescription 33 | 0.511 | 0.623 |
| Prescription 55 | 0.478 | 0.602 |
| Prescription 56 | 0.532 | 0.735 |
| Prescription 58 | 0.489 | 0.630 |
| Prescription 59 | 0.536 | 0.682 |
| Comparative prescription 2 | 0.608 | 7.402 |
| Comparative prescription 3 | 0.503 | 6.239 |
| Comparative prescription 3 | 0.507 | 7.783 |
| Comparative prescription 4 | 0.667 | 3.452 |
| Comparative prescription 5 | 0.500 | 6.239 |
| Comparative prescription 6 | 9.405 | 19.651 |
| Comparative prescription 7 | 1.326 | 12.303 |
| Comparative prescription 8 | 0.724 | 9.700 |
| Comparative prescription 9 | 11.380 | 6.761 |
| Comparative prescription 10 | 0.507 | 7.783 |
| Comparative prescription 11 | 11.324 | — |
| Comparative prescription 12 | 13.401 | — |

Result analysis: The particle size of apalutamide-nanocrystalline obtained by the prescription and preparation methods described in prescriptions 1-6, 8-17, 19-22, 24-29, 31-34, 55-56, and 58-59 did not change significantly before and after curing, while the particle size of apalutamide nanocrystals obtained by comparison prescriptions 2-5, 8 and 10 increased significantly after curing, and the particle size of apalutamide nanocrystals obtained by prescriptions 7, 9, 11 and 12 was large after milling, indicating that the ideal particle size or particle size stability of apalutamide nanocrystals could not be obtained when there was no spatial stabilizer, or the amount and type of spatial stabilizer and protective agent were unreasonable.

### Example 31: Pharmacokinetic Studies in Vivo in Animals

Dosing regimen: Two preparations and two crosses were used (8 healthy Beagle dogs, half male and half female, were divided into 2 groups, 4 in each group).

Experimental group: Apalutamide nanocrystalline tablets (specification: 60 mg) obtained from prescription 46 in example 19 once a day.

Control group: Apalutamide tablets (non-milling; specification: 60 mg) obtained from comparative prescription 1 in comparative example 1 once a day.

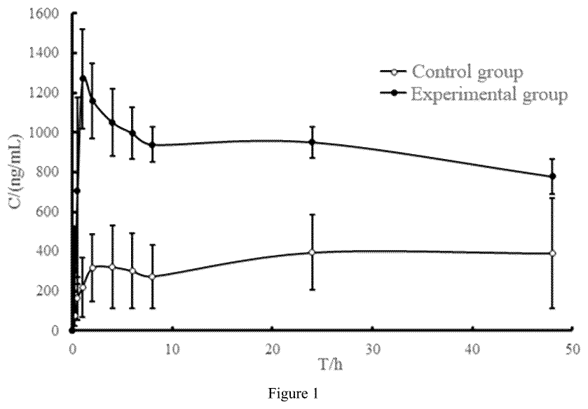

After administration under fasting conditions, blood samples were collected at 0, 0.5, 1, 2, 4, 6, 8, 24 and 48 hours to determine the blood concentration, and the drug time curve was plotted, see FIG. 1.

Table 34 listed the pharmacokinetic data of 60 mg apalutamide tablets prepared by prescription 46 and comparative prescription 1 in Beagle dogs.

TABLE 34
Results of Pharmacokinetic Studies in Vivo in Animals

| | The highest blood | Drug-time curve area |
|---|---|---|
| | drug concentration | from 0 to 48 hours |
| Sample | Cmax (ng/mL) | AUC (h*ng/ml) |
| Prescription 46 | 1230 ± 450 | 121000 ± 29000 |
| Comparative | 498 ± 250 | 40700 ± 20000 |
| prescription 1 | | |

Result analysis: The apalutamide nanocrystalline composition after milling had a faster and higher blood drug concentration in the body, the highest blood drug concentration was 2.47 times that of the unmilled apalutamide composition, and the 0-48 hours drug-time curve area was 3 times that of the unmilled apalutamide composition, indicating that the apalutamide nanocrystalline composition after milling could effectively improve the bioavailability of apalutamide.

### Example 32: Pharmacokinetic Studies in Vivo in Animals

Dosing regimen: Three preparations and three crosses were used (12 healthy Beagle dogs, half male and half female, were divided into 3 groups, 4 in each group).

Experimental group: group A: Apalutamide nanocrystalline tablets (specification: 60 mg) obtained from prescription 46 in example 19, once a day.

group B: Apalutamide nanocrystalline tablets (specification: 60 mg) obtained from prescription 47 in example 19, once a day.

Control group: group C: The reference listed drug (the manufacturer is Xi′an Janssen, the specification is 60 mg, and the batch number is 19LG0585) apalutamide tablets (specification: 60 mg), once a day.

Because the half-life of the product is too long, the design of the blood collection point of the first animal experiment is not long enough (see example 31), so the blood collection time of the animal experiment design is longer.

After administration under fasting conditions, blood samples were collected at 0, 1, 2, 4, 6, 8, 24, 48, 96 and 144 hours to determine the blood concentration.

Table 35 listed the pharmacokinetic data of 60 mg apalutamide tablets and reference listed drug prepared by prescription 46 and 47 in Beagle dogs.

TABLE 35
Results of Pharmacokinetic Studies in Vivo in Animals

| | The highest blood | Drug-time curve area |
|---|---|---|
| | drug concentration | from 0 to 144 hours |
| Sample | Cmax (ng/mL) | AUC(h*ng/ml) |
| Group A: Prescription 46 | 1080 ± 690 | 118000 ± 68000 |
| Group B: Prescription 47 | 2160 ± 1090 | 178000 ± 116000 |
| Group C: The reference | 2910 ± 1420 | 228000 ± 158000 |
| listed drug | | |
| Prescription 46/RLD ratio | 37.11% | 51.75% |
| Prescription 47/RLD ratio | 74.23% | 78.07% |

Result analysis: According to the results of the pharmacokinetic studies in vivo of the animals in Table 34, this animal experiment adopts a three-crossover experimental design, because the number of animals is only 12, the variation of Cmax and AUC is large, more than 50%, and the inventor believes that the relative bioavailability is greater than 70%, and there is no significant difference between prescription 47 and RLD. The highest blood drug concentration and drug-time curve area from 0 to 144 hours of prescription 46 were 37.11% and 51.75% of the reference time, respectively, and the bioavailability was significantly lower than that of the reference listed drug, and the efficacy of taking the preparation was not guaranteed. The highest blood drug concentration of prescription 47 and drug-time curve area from 0 to 144 hours of drug time were 74.23% and 78.07% of the reference respectively, which were higher than 70%, and the bioavailability was closer to the reference listed drug, and there was no significant difference; compared with prescription 47 and prescription 46, the bioavailability increased substantially, and compared with the comparative prescription 1 in example 31, the bioavailability increased several times. Using the conventional dissolution detection method, there was little difference in the dissolution of the two compositions of prescription 46 and prescription 47, but the absorption in animals showed a large difference, and the effect of prescription 47 was better.

The inventors believe that when the bioavailability test is carried out in animals, the number of animals generally selected is limited, the experimental data will be relatively variable, and the significance of statistical analysis of confidence interval is limited, and it is more statistically significant to use the mean comparison to determine the relative bioavailability. The inventors believe that when the bioavailability test is carried out in animals, significant differences in relative bioavailability are considered to be significant if there is a difference in the magnitude level, such as a 1-fold increase (or 50% reduction) in bioavailability, a 2-fold increase (or 66% reduction), etc. For drugs with large differences, EMEA broadens the Cmax standard of bioequivalence to 69.84%-143.19%. Due to the shortcomings of animal experiments, such as the relatively small number of experimental cases and large differences, the inventors believe that the relative bioavailability of the developed product is in the range of 70%-143%, and it can be considered that there is no significant difference in the bioavailability of the product.

### Example 33: Prescription 46, Prescription 47 and Reference Listed Drug were Investigated for Different Pore Sizes

The animal experimental data in Example 32 show that the absorption of prescription 46 and prescription 47 was very different in vivo, while the dissolution in vitro was similar. Therefore, it is considered that the existing in vitro dissolution method is not related to in vivo absorption, and the method of dissolution method is investigated again. The apalutamide nanocrystalline tablets prepared by prescription 46 and prescription 47 in example 19 were tested in vitro dissolution by paddle method under the conditions of pH 6.8, volume of dissolution medium 900 mL, temperature of 37.0±0.5° C., and 75 rpm, and the sampling time points of dissolution were 5 min, 10 min, 15 min, 20 min, 30 min and 60 min; Sampling location: from the top of the paddle blade to the midpoint of the liquid level, 10 mm away from the inner wall of the dissolution cup, 0.45 μm PES and 0.22 μm PES membrane filtration were used to determine the dissolution content in vitro by high-performance liquid chromatography method. The test results are shown in Table 36.

TABLE 36
Dissolution Investigation Under Different Membrane Pore Sizes

| Prescription | Membrane | 5 | 10 | 15 | 20 | 30 | 60 |
|---|---|---|---|---|---|---|---|
| number | model | min | min | min | min | min | min |
| Prescription | 0.45 μm PES | 43 | 79 | 92 | 92 | 93 | 96 |
| 46 | 0.22 μm PES | 37 | 76 | 93 | 93 | 93 | 93 |
| Prescription | 0.45 μm PES | 42 | 76 | 92 | 94 | 93 | 94 |
| 47 | 0.22 μm PES | 35 | 74 | 95 | 94 | 95 | 91 |
| Reference | 0.45 μm PES | 57 | 67 | 70 | 74 | 78 | 83 |
| listed drug | 0.22 μm PES | 57 | 65 | 71 | 73 | 79 | 82 |

Result analysis: According to the dissolution results in Table 36, there was no difference in the dissolution of prescriptions 46 and 47 for filter membranes with different pore sizes, which were higher than those of the reference listed drug, and the method was not related in vitro and in vivo, and other dissolution methods need to be investigated.

### Example 34: The Dissolution was Determined by In-Situ Optical Fiber of Prescription 46, Prescription 47 and Reference Listed Drug

The apalutamide nanocrystalline tablets prepared by prescription 46 and prescription 47 in example 19 were tested by paddle method under the conditions of pH 6.8, volume of dissolution medium 900 mL, temperature of 37.0±0.5° C., and 50 rpm, and the in situ optical fiber was used to determine the dissolution, the wavelength range was 252-267 nm, and the 5 mm optical cell was selected. The results are shown in table 37.

TABLE 37
In-Situ Fiber Dissolution Data

| Prescription | | | | | | |
|---|---|---|---|---|---|---|
| number | 5 min | 10 min | 15 min | 20 min | 30 min | 60 min |
| Prescription 46 | 26 | 43 | 42 | 42 | 43 | 44 |
| Prescription 47 | 25 | 50 | 60 | 62 | 62 | 63 |
| Reference | 59 | 77 | 83 | 84 | 81 | 74 |
| listed drug | | | | | | |

Result analysis: According to the dissolution results in Table 37, the dissolution results of prescription 46 and 47 were different from those of the reference listed drug by in-situ optical fiber determination, and the results were close to those in vivo, and the dissolution method had a good correlation in vitro and in vivo.

### Example 35: The Dissolution was Determined Using an In-Situ Optical Fiber

Animal PK experiments confirmed that the dissolution method using in-situ fiber had a stronger correlation with in vivo absorption, and the dissolution results measured by the high-performance liquid phase method had a slightly worse correlation with in vivo absorption. Therefore, the apalutamide oral nanocrystalline tablets prepared from prescriptions 1-6, 8-17, 19-22, 24-29, 31-34, 55-56, 58-59 and comparative prescriptions 2-12 in the above examples were taken and dissolved by in-situ optical fiber, and the test results are shown in Table 38.

The results of dissolution showed that the combination of steric hindrance protector polymer HPC and ionic surfactant had a more significant solubilization effect, which was more than 4 times higher than that of the non-solubilizing API (comparative prescription 1), which was closer to RLD. When PVP was used as a spatial stabilizer, it was 3 times higher than that of non-soluble API.

TABLE 38
Dissolution Results of Each Prescription Determined
by In-Situ Fiber Optics

| Prescription | | | | | | |
|---|---|---|---|---|---|---|
| number | 5 min | 10 min | 15 min | 20 min | 30 min | 60 min |
| Prescription 1 | 21 | 37 | 46 | 49 | 49 | 48 |
| Prescription 2 | 25 | 50 | 61 | 62 | 62 | 64 |
| Prescription 3 | 20 | 49 | 60 | 63 | 64 | 65 |
| Prescription 4 | 22 | 30 | 46 | 48 | 49 | 50 |
| Prescription 5 | 20 | 44 | 62 | 64 | 64 | 64 |
| Prescription 6 | 25 | 46 | 63 | 65 | 65 | 65 |
| Prescription 8 | 32 | 54 | 61 | 62 | 61 | 61 |
| Prescription 9 | 41 | 61 | 61 | 61 | 64 | 60 |
| Prescription 10 | 48 | 60 | 61 | 62 | 62 | 60 |
| Prescription 11 | 29 | 52 | 63 | 63 | 64 | 65 |
| Prescription 12 | 31 | 52 | 59 | 63 | 63 | 63 |
| Prescription 13 | 14 | 37 | 40 | 41 | 43 | 44 |
| Prescription 14 | 19 | 32 | 42 | 42 | 43 | 45 |
| Prescription 15 | 20 | 36 | 43 | 44 | 45 | 47 |
| Prescription 16 | 26 | 37 | 45 | 46 | 47 | 47 |
| Prescription 17 | 30 | 35 | 40 | 44 | 43 | 43 |
| Prescription 19 | 20 | 35 | 44 | 49 | 48 | 48 |
| Prescription 20 | 19 | 34 | 42 | 47 | 44 | 47 |
| Prescription 21 | 21 | 36 | 42 | 44 | 44 | 45 |
| Prescription 22 | 24 | 35 | 41 | 45 | 45 | 45 |
| Prescription 24 | 40 | 58 | 57 | 59 | 61 | 66 |
| Prescription 25 | 17 | 44 | 61 | 62 | 65 | 63 |
| Prescription 26 | 12 | 20 | 23 | 24 | 25 | 25 |
| Prescription 27 | 28 | 42 | 43 | 44 | 46 | 47 |
| Prescription 28 | 25 | 43 | 46 | 45 | 44 | 45 |
| Prescription 29 | 27 | 47 | 47 | 48 | 47 | 47 |
| Prescription 32 | 31 | 52 | 64 | 66 | 66 | 66 |
| Prescription 33 | 35 | 48 | 67 | 67 | 67 | 66 |
| Prescription 34 | 39 | 55 | 65 | 65 | 65 | 65 |
| Prescription 35 | 33 | 48 | 63 | 63 | 63 | 63 |
| Prescription 36 | 25 | 42 | 57 | 63 | 63 | 64 |
| Prescription 37 | 41 | 56 | 67 | 67 | 69 | 69 |
| Prescription 40-1 | 22 | 58 | 65 | 66 | 66 | 66 |
| Prescription 40-2 | 29 | 60 | 66 | 66 | 66 | 66 |
| Prescription 40-3 | 32 | 62 | 67 | 67 | 67 | 68 |
| Prescription 43-1 | 41 | 48 | 56 | 65 | 66 | 66 |
| Prescription 43-2 | 39 | 46 | 57 | 64 | 66 | 66 |
| Prescription 43-3 | 36 | 42 | 52 | 60 | 65 | 65 |
| Prescription 43-4 | 20 | 26 | 30 | 33 | 40 | 40 |
| Prescription 47 | 32 | 56 | 62 | 64 | 64 | 65 |
| Prescription 49 | 16 | 29 | 56 | 64 | 65 | 66 |
| Prescription 50 | 64 | 65 | 65 | 67 | 67 | 67 |
| Prescription 55 | 22 | 40 | 52 | 63 | 63 | 62 |
| Prescription 56 | 39 | 54 | 59 | 64 | 65 | 65 |
| Prescription 58 | 30 | 50 | 64 | 65 | 65 | 66 |
| Prescription 59 | 23 | 41 | 56 | 60 | 62 | 62 |
| Comparative | 7 | 8 | 10 | 11 | 12 | 15 |
| prescription 1 | | | | | | |
| Comparative | 9 | 10 | 17 | 23 | 34 | 34 |
| prescription 2 | | | | | | |
| Comparative | 7 | 14 | 21 | 24 | 25 | 22 |
| prescription 3 | | | | | | |
| Comparative | 7 | 14 | 19 | 19 | 20 | 22 |
| prescription 4 | | | | | | |
| Comparative | 6 | 8 | 9 | 9 | 9 | 11 |
| prescription 5 | | | | | | |
| Comparative | 7 | 10 | 18 | 21 | 25 | 25 |
| prescription 6 | | | | | | |
| Comparative | 12 | 19 | 24 | 26 | 29 | 28 |
| prescription 7 | | | | | | |
| Comparative | 12 | 14 | 15 | 16 | 16 | 16 |
| prescription 10 | | | | | | |
| Comparative | 10 | 11 | 19 | 18 | 19 | 19 |
| prescription 11 | | | | | | |
| Comparative | 9 | 14 | 20 | 21 | 20 | 21 |
| prescription 12 | | | | | | |
| Comparative | 12 | 16 | 19 | 20 | 24 | 27 |
| prescription 15 | | | | | | |
| Reference listed | 59 | 77 | 83 | 84 | 81 | 74 |
| drug | | | | | | |
| (19LG0585) | | | | | | |

### Example 36: Screening of Spatial Stabilizer Types

Spatial stabilizers other than the above-mentioned polymer materials were examined, and the composition prescription was shown in Table 39.

TABLE 39
Prescription Table for Screening Types of Spatial Stabilizer

| | | Single dose |
|---|---|---|
| | | mg/tablet |
| Component | | Prescription 60 |
| Active ingredient | Apalutamide | 60 |
| Stabilizer | Hydroxypropyl methylcellulose | 30 |
| | acetate succinate | |
| Ionic surfactant | Sodium dodecyl sulfate | 6 |
| Protective agent | Mannitol | 78 |
| Additional excipients | Mannitol | 104.1 |
| | Croscarmellose sodium | 15 |
| | Micronized silica gel | 3.9 |
| | Magnesium stearate | 3 |

| | |
|---|---|
| Total | 300 |

### Preparation Process:

According to Table 39, the prescribed amount of stabilizer hydroxypropyl methylcellulose acetate succinate and sodium dodecyl sulfate were weighed, 6.8 medium was added and the mixture was stirred until the solution was clear, and then the prescribed amount of apalutamide was added and the mixture was stirred stir to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 37: The Dissolution was Determined Using an In-Situ Optical Fiber

The apalutamide oral nanocrystalline tablets prepared from prescriptions 1-6, 60 and comparative prescriptions 1-5 in the above examples were taken and dissolved by in-situ optical fiber, and the test results are shown in Table 40.

TABLE 40
Dissolution Results Determined by In-Situ Fiber Optics

| Prescription | | | | | | |
|---|---|---|---|---|---|---|
| number | 5 min | 10 min | 15 min | 20 min | 30 min | 60 min |
| Prescription 1 | 21 | 37 | 46 | 49 | 49 | 48 |
| Prescription 2 | 25 | 50 | 61 | 62 | 62 | 64 |
| Prescription 3 | 20 | 49 | 60 | 63 | 64 | 65 |
| Prescription 4 | 22 | 30 | 46 | 48 | 49 | 50 |
| Prescription 5 | 20 | 44 | 62 | 64 | 64 | 64 |
| Prescription 6 | 25 | 46 | 63 | 65 | 65 | 65 |
| Prescription 60 | 30 | 54 | 65 | 70 | 72 | 72 |
| Comparative | 7 | 8 | 10 | 11 | 12 | 15 |
| prescription 1 | | | | | | |
| Comparative | 9 | 10 | 17 | 23 | 34 | 34 |
| prescription 2 | | | | | | |
| Comparative | 7 | 14 | 21 | 24 | 25 | 22 |
| prescription 3 | | | | | | |
| Comparative | 7 | 14 | 19 | 19 | 20 | 22 |
| prescription 4 | | | | | | |
| Comparative | 6 | 8 | 9 | 9 | 9 | 11 |
| prescription 5 | | | | | | |
| Reference listed | 59 | 77 | 83 | 84 | 81 | 74 |
| drug | | | | | | |
| (19LG0585) | | | | | | |

Under the same conditions, the apalutamide nanocrystalline tablets of prescriptions 1˜6 and prescription 60 using povidone K17, hydroxypropyl cellulose, vinylpyrrolidone-vinyl acetate copolymer, polyvinyl alcohol, hydroxypropyl methylcellulose E5, hydroxypropyl methylcellulose E3 as spatial stabilizers had more higher dissolution rate and faster dissolution speed in pH 6.8 medium than the apalutamide nanocrystalline tablets with comparative prescription 2˜5 using Soluplus, Tween-80, Poloxamer P188 and polyethylene glycol 6000 as spatial stabilizers. In addition, compared with other types of stabilizers, the stabilizers povidone K17 and polyvinyl alcohol were about 15% lower than those of other optical fibers. Spatial stabilizers preferably at least one of hydroxypropyl cellulose, vinylpyrrolidone-vinyl acetate copolymer, hydroxypropyl methylcellulose E5, hydroxypropyl methylcellulose E3, and hydroxypropyl methylcellulose acetate succinate could increase dissolution to more than 4 times that of ordinary APIs.

### Example 38: Screening of the Proportion of Ionic Surfactant (HPC)

HPC was used as a spatial stabilizer to confirm the effect of different surface activity concentrations on dissolution. Composition prescription: as shown in Table 41.

TABLE 41
Screening Prescription Table of Ionic Surfactant Ratio

| | | Single dose mg/tablet | | | | | | |
|---|---|---|---|---|---|---|---|---|
| | | | | | | | Comparative | Comparative |
| | | Prescription | Prescription | Prescription | Prescription | Prescription | prescription | prescription |
| Component | | 61 | 62 | 63 | 64 | 65 | 16 | 17 |
| Active | Apalutamide | 60 | 60 | 60 | 60 | 60 | 60 | 60 |
| ingredient | | | | | | | | |
| Spatial | Hydroxypropyl | 17.14 | 17.14 | 17.14 | 17.14 | 17.14 | 17.14 | 17.14 |
| stabilizer | cellulose | | | | | | | |
| Ionic | Sodium | 30 | 12 | 3 | 0.3 | 0.15 | — | 60 |
| surfactant | dodecyl | | | | | | | |
| | sulfate | | | | | | | |
| Protective | Mannitol | 78 | 78 | 78 | 78 | 78 | 78 | 78.00 |
| agent | | | | | | | | |
| Additional | Mannitol | 92.96 | 110.96 | 119.96 | 122.66 | 122.81 | 122.96 | 62.96 |
| excipients | Croscarmellose | 15 | 15 | 15 | 15 | 15 | 15 | 15.00 |
| | sodium | | | | | | | |
| | Micronized | 3.9 | 3.9 | 3.9 | 3.9 | 3.9 | 3.9 | 3.90 |
| | silica gel | | | | | | | |
| | Magnesium | 3 | 3 | 3 | 3 | 3 | 3 | 3.00 |
| | stearate | | | | | | | |

| | | | | | | | |
|---|---|---|---|---|---|---|---|
| Total | 300 | 300 | 300 | 300 | 300 | 300 | 300 |

### Preparation Process:

According to Table 41, the prescribed amount of spatial stabilizer hydroxypropyl cellulose and sodium dodecyl sulfate were weighed, water was added and stirred until the solution was clear, and then the prescribed amount of apalutamide was added and the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

Comparative Prescription 17 produced a large number of air bubbles during the milling process and was unable to mill to nanoscale particles.

### Example 39: The Dissolution was Determined Using an In-Situ Optical Fiber

The apalutamide oral nanocrystalline tablets prepared from prescriptions 62-65 and comparative prescriptions 1-5 in the above examples were taken and dissolved by in-situ optical fiber, and the test results are shown in Table 42.

TABLE 42
Dissolution Results Determined by In-Situ Fiber Optics

| Prescription | | | | | | |
|---|---|---|---|---|---|---|
| number | 5 min | 10 min | 15 min | 20 min | 30 min | 60 min |
| Prescription 61 | 31 | 52 | 63 | 64 | 65 | 65 |
| Prescription 62 | 35 | 50 | 64 | 65 | 65 | 66 |
| Prescription 11 | 29 | 52 | 63 | 63 | 64 | 65 |
| Prescription 63 | 28 | 49 | 62 | 63 | 63 | 63 |
| Prescription 64 | 30 | 50 | 63 | 64 | 64 | 64 |
| Prescription 65 | 25 | 30 | 30 | 32 | 33 | 33 |
| Comparative | 10 | 14 | 22 | 23 | 25 | 25 |
| prescription 16 | | | | | | |
| Comparative | 12 | 13 | 14 | 15 | 16 | 17 |
| prescription 17 | | | | | | |
| Reference listed | 59 | 77 | 83 | 84 | 81 | 74 |
| drug (19LG0585) | | | | | | |

Result analysis: The above in-situ optical fiber dissolution results show that when the weight ratio of sodium dodecyl sulfate as an ionic surfactant to apalutamide was in the range of 1:200-1:2, the obtained apalutamide nanocrystalline tablets had high dissolution rate and fast dissolution speed in pH 6.8 medium, not that the higher the amount of surfactant, the higher the dissolution. In the composition of apalutamide nanocrystals, the ionic surfactant mainly achieves the stability effect between particles in the system through the potential effect, and the addition of too much ionic surfactant will lead to a large number of bubbles in the milling process, which is not conducive to milling, and may also lead to flocculation, and excessive dosage is easy to cause toxicity problems; therefore, the preferred range of ionic surfactant is that the weight ratio of ionic surfactant to apalutamide is 1:200-1:2.

### Example 40: Screening of the Rate of Protective Agent

The in-situ optical fiber was used to determine the dissolution, and the dosage of different curing protective agents had a great influence on the dissolution, mannitol was used as the protective agent in prescription 26, and the dosage was ¼ of the active ingredient (API), and the dissolution was greatly reduced, and the proportion of protective agents was supplemented. Composition prescription: as shown in Table 43.

TABLE 43
Screening Prescription Table of Protective Agent Ratio

| Component | | Prescription 66 |
|---|---|---|
| Active ingredient | Apalutamide | 60 |
| Spatial stabilizer | Hydroxypropyl cellulose | 6 |
| Ionic surfactant | Sodium dodecyl sulfate | 6 |
| Protective agent | Mannitol | 30 |
| Additional excipients | Mannitol | 176.10 |
| | Croscarmellose sodium | 15 |
| | Micronized silica gel | 3.9 |
| | Magnesium stearate | 3 |

| | |
|---|---|
| Total | 300 |

Preparation process: According to Table 43, the prescribed amount of spatial stabilizer and sodium dodecyl sulfate were weighed, water was added and stirred until the solution was clear, and then the prescribed amount of apalutamide was added and the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 41: Dissolution Test

The apalutamide oral nanocrystalline tablets prepared from prescriptions 11, 24-26, 66 and comparative prescription 10 in the above examples were taken and dissolved by in-situ optical fiber, and the test results are shown in Table 44.

TABLE 44
Dissolution Results of Prescriptions 11, 24-26, 66 and
Comparative Prescription 10 in pH 6.8 Medium
(%, n = 3)

| Prescription | | | | | | |
|---|---|---|---|---|---|---|
| number | 5 min | 10 min | 15 min | 20 min | 30 min | 60 min |
| Prescription 24 | 31 | 55 | 65 | 65 | 66 | 66 |
| Prescription 11 | 29 | 52 | 63 | 63 | 64 | 65 |
| Prescription 25 | 26 | 50 | 64 | 64 | 64 | 66 |
| Prescription 66 | 28 | 48 | 60 | 60 | 60 | 60 |
| Prescription 26 | 12 | 20 | 23 | 24 | 25 | 25 |
| Comparative | 12 | 16 | 15 | 17 | 16 | 17 |
| prescription 10 | | | | | | |
| Reference listed | 59 | 77 | 83 | 84 | 81 | 74 |
| drug | | | | | | |
| (19LG0585) | | | | | | |

Result analysis: The above dissolution results showed that the weight ratio of the protective agent to apalutamide in the range of 8:1˜1:2 obtained apalutamide nanocrystalline tablets had high dissolution rate and fast dissolution speed in pH 6.8 medium.

In the apalutamide nanocrystal composition, it is important to add a suitable protective agent in order to reduce the damage during the solidification of the nanocrystals and maintain the redispersion property of the nanocrystals. Too much protective agent will lead to too much tablet weight; too little or no protectant protective agent, and the water will be lost instantaneously during the curing process, which will lead to the destruction of the nanocrystalline structure, easy to coalescence between particles, and poor redispersibility, which is not conducive to the physical stability of apalutamide nanocrystalline composition. In the present invention, the weight ratio of the protective agent to apalutamide is preferably 8:1˜1:2, which can not only achieve the technical effect of high dissolution rate and fast dissolution speed, but also reduce the weight of the tablet and improve the physical stability of the apalutamide nanocrystalline composition.

### Example 42: Screening of Surfactant Species

Composition prescription: as shown in Table 45.

TABLE 45
Screening Prescription Table of Surfactant Species

| | | Single dose mg/tablet | | | | |
|---|---|---|---|---|---|---|
| | | Prescription | Prescription | Prescription | Prescription | Prescription |
| Component | | 67 | 68 | 69 | 70 | 71 |
| Active | Apalutamide | 60 | 60 | 60 | 60 | 60 |
| ingredient | | | | | | |
| Spatial | Hydroxypropyl | 6 | 6 | 6 | 6 | 6 |
| stabilizer | cellulose | | | | | |
| Non-ionic | Tween80 | 6 | — | — | — | — |
| surfactants | Tween20 | — | 6 | — | | |
| | TPGS | — | — | 6 | | |
| | Poloxamer 188 | — | — | — | 6 | — |
| | Poloxamer 407 | — | — | — | | 6 |
| Protective | Mannitol | 78 | 78 | 78 | 78 | 78 |
| agent | | | | | | |
| Additional | Mannitol | 128.1 | 128.1 | 128.1 | 128.1 | 128.1 |
| excipients | Croscarmellose sodium | 15 | 15 | 15 | 15 | 15 |
| | Micronized silica gel | 3.9 | 3.9 | 3.9 | 3.9 | 3.9 |
| | Magnesium stearate | 3 | 3 | 3 | 3 | 3 |

| | | | | | |
|---|---|---|---|---|---|
| Total | 300 | 300 | 300 | 300 | 300 |

### Preparation Process:

According to Table 45, the prescribed amount of hydroxypropyl cellulose and surfactants were weighed, water was added and stirred until the solution was clear, and then the prescribed amount of apalutamide was added and the mixture was stirred to obtain a suspension; milling medium and suspension were added to the milling chamber for milling to obtain a nanocrystalline suspension; the nano suspension was mixed with the prescription amount of protective agent, spray dried; the prescribed amount of additional excipients mannitol, croscarmellose sodium and micronized silica gel were added and the mixture was mixed, and then magnesium stearate was added, mixed and compressed, then apalutamide nanocrystalline tablets were obtained.

### Example 43: Dissolution Test

The apalutamide oral nanocrystalline tablets prepared from prescriptions 11, 55-56, 67-71 and comparative prescriptions 11-12 in the above examples were taken and the dissolution was determined by in-situ optical fiber, and the test results are shown in Table 46.

TABLE 46
Dissolution Results of Prescriptions 11, 55-56, 67-71 and
Comparative Prescriptions 11-12 in pH 6.8 Medium
(%, n = 3)

| Prescription | | | | | | |
|---|---|---|---|---|---|---|
| number | 5 min | 10 min | 15 min | 20 min | 30 min | 60 min |
| Prescription 11 | 29 | 52 | 63 | 63 | 64 | 65 |
| Prescription 55 | 28 | 50 | 62 | 63 | 63 | 63 |
| Prescription 56 | 23 | 45 | 55 | 56 | 58 | 58 |
| Prescription 67 | 19 | 32 | 40 | 41 | 42 | 43 |
| Prescription 68 | 20 | 30 | 39 | 40 | 40 | 40 |
| Prescription 69 | 18 | 22 | 29 | 30 | 30 | 30 |
| Prescription 70 | 15 | 25 | 30 | 35 | 36 | 35 |
| Prescription 71 | 16 | 23 | 32 | 34 | 34 | 34 |
| Comparative | 10 | 11 | 19 | 18 | 19 | 19 |
| prescription 11 | | | | | | |
| Comparative | 9 | 14 | 20 | 21 | 20 | 21 |
| prescription 12 | | | | | | |
| Reference listed | 59 | 77 | 83 | 84 | 81 | 74 |
| drug (19LG0585) | | | | | | |

Result analysis: Using the polymer material hydroxypropyl cellulose as a spatial stabilizer, and adding different types of surfactants respectively, the inventors were pleasantly surprised to find that the ionic surfactant is far better than other types of surfactants (see the prescription of example 42). Among them, the solubilization effect of sodium dodecyl sulfate or sodium multicuronate is better, and the solubilization effect of lecithin as surfactant is also significantly improved.

### Example 44: Particle Size Distribution

The nanocrystalline suspension of prescription 67-71 prepared by the above-mentioned examples and the powder obtained after spray drying were taken, and the particle size distribution of the nanocrystalline suspension after milling (before curing) and the powder obtained after spray drying after re-dispersion (the suspension after curing) was determined by a laser particle size analyzer, and the test results are shown in Table 47. The inventors were pleasantly surprised to find that with a nonionic surfactant composition, the cured and redispersed suspension was still a nanoscale suspension; it can be seen that the solubilization effect of the composition of other types of surfactants other than ionic surfactants was still not good, which was not caused by the instability of particle size, but by the special properties of the surfactant itself, and the use of ionic surfactants was conducive to improving the dissolution of apalutamide compositions.

TABLE 47
Particle Size Distribution

| | | Particle size of the |
|---|---|---|
| | Particle size before | redispersed suspension |
| | curing (μm) | after curing (μm) |
| Prescription number | D90 | D90 |
| Prescription 67 | 0.579 | 0.582 |
| Prescription 68 | 0.569 | 0.573 |
| Prescription 69 | 0.700 | 0.711 |
| Prescription 70 | 0.568 | 0.570 |
| Prescription 71 | 0.600 | 0.610 |

### Example 45: Curing Process—Drug-Coated Pellets

Sucrose was used as the protective agent and the drug-coated pellets process was used to apply the apalutamide nanocrystalline suspension on the sucrose pill core, and the effects of different drug loading on the dissolution were investigated. Composition prescription: as shown in Table 48.

TABLE 48
Screening Prescription Table of Curing Process in Apalutamide Nanocrystalline Tablets

| | | | | | | Single dose |
|---|---|---|---|---|---|---|
| | | | | | | mg/capsule |
| | | Single dose mg/capsule | | | | Reference |
| | | Prescription | Prescription | Prescription | Prescription | listed drug |
| Component | | 72-1 | 72-2 | 72-3 | 72-4 | (19LG0585) |
| Active ingredient | Apalutamide | 60 | 60 | 60 | 60 | 60 |
| Spatial stabilizer | Hydroxypropyl | 6 | 6 | 6 | 6 | Excipient |
| | cellulose | | | | | 640 mg |
| Ionic surfactant | Sodium dodecyl | 6 | 6 | 6 | 6 | |
| | sulfate | | | | | |
| Protective agent | Cane sugar | 60 | 60 | 60 | 60 | |
| Substrate | Sucrose pill core | 132 | 66 | 40 | 18 | |

| | | | | | |
|---|---|---|---|---|---|
| Medicated weight gain | 100% | 200% | 330% | 733% | |
| Total (mg) | 264 | 198 | 172 | 150 | 700 mg |
| Drug loading | 22.7% | 30.3% | 34.9% | 40.0% | 8.57% |

### Preparation Process:

According to Table 48, the prescribed amount of spatial stabilizer and sodium dodecyl sulfate were weighed, water was added and stirred until the solution was clear, and then the prescribed amount of apalutamide was added and the mixture was stirred to obtain a suspension; milling medium and suspension were added to the milling chamber for milling to obtain a nanocrystalline suspension; the nano suspension was mixed with the prescription amount of protective agent, the pellets were applied with drug, and the weight was increased by different multiples to obtain different drug loading pills, which were directly filled with capsules.

### Example 46: Dissolution Test

The apalutamide oral nanocrystal tablets prepared from prescriptions 25, 32, 33-37, 47, 49, 50, 40-1, 40-2, 40-3, 43-1, 43-2, 43-3, 72-1, 72-2 and 72-3 were taken and the dissolution was determined by in-situ optical fiber, and the test results are shown in Table 49.

TABLE 49
Dissolution Results of Each Prescription (%, n = 3)

| Prescription | | | | | | |
|---|---|---|---|---|---|---|
| number | 5 min | 10 min | 15 min | 20 min | 30 min | 60 min |
| Prescription 25 | 26 | 48 | 54 | 64 | 64 | 64 |
| Prescription 32 | 31 | 52 | 64 | 66 | 66 | 66 |
| Prescription 33 | 35 | 48 | 67 | 67 | 67 | 66 |
| Prescription 34 | 39 | 55 | 65 | 65 | 65 | 65 |
| Prescription 35 | 33 | 48 | 63 | 63 | 63 | 63 |
| Prescription 36 | 25 | 42 | 57 | 63 | 63 | 64 |
| Prescription 37 | 41 | 56 | 67 | 67 | 69 | 69 |
| Prescription 72-2 | 52 | 56 | 66 | 68 | 69 | 69 |
| Prescription 72-1 | 45 | 50 | 65 | 66 | 68 | 68 |
| Prescription 72-3 | 55 | 58 | 66 | 68 | 68 | 68 |
| Prescription 72-4 | 60 | 66 | 68 | 69 | 69 | 69 |
| Prescription 40-1 | 22 | 58 | 65 | 66 | 66 | 66 |
| Prescription 40-2 | 29 | 60 | 66 | 66 | 66 | 66 |
| Prescription 40-3 | 32 | 62 | 67 | 67 | 67 | 68 |
| Prescription 43-1 | 41 | 48 | 56 | 65 | 66 | 66 |
| Prescription 43-2 | 39 | 46 | 57 | 64 | 66 | 66 |
| Prescription 43-3 | 36 | 42 | 52 | 60 | 65 | 65 |
| Prescription 43-4 | 20 | 26 | 30 | 33 | 40 | 40 |
| Prescription 47 | 32 | 56 | 62 | 64 | 64 | 65 |
| Prescription 49 | 16 | 29 | 56 | 64 | 65 | 66 |
| Prescription 50 | 64 | 65 | 65 | 67 | 67 | 67 |
| Reference | 59 | 77 | 83 | 84 | 81 | 74 |
| listed drug | | | | | | |
| (19LG0585) | | | | | | |

Result analysis: The above dissolution results show that by adopting the prescription described in the present invention, the curing process adopted spray drying, fluidized bed spray drying, freeze drying or drug-coated pellets, and the apalutamide preparation with fast dissolution speed could be obtained.

In the curing process of fluidized bed spraying, mannitol, microcrystalline cellulose, microcrystalline cellulose pellet core or/and anhydrous calcium hydrogen phosphate were used as protective agents, and the dissolution was relatively fast.

The drug loading capacity of apalutamide nanocrystal composition can be as high as 40%, which can maintain a high dissolution, and can greatly reduce the weight and size of the preparation product in the case of high drug loading, and avoid the problem of swallowing difficulties caused by excessive size. The reference listed drug is less than 10% loaded and requires 4 tablets to be taken at the same time, which further increases the difficulty of the patient to take the drug and increases the probability of the patient taking the drug incorrectly. The nanocrystalline composition provided by the present invention can greatly increase the drug loading, greatly reduce the weight and size of the preparation, and can prepare the preparation product of 120 mg, 240 mg specification of appropriate size simultaneously, increase the patient's medication compliance, and reduce the difficulty of the patient's medication management.

### Example 28: Investigation of the Particle Size of Ball Mills

The suspension was prepared as prescribed in Table 50:

TABLE 50
Particle Size Investigation of Apalutamide
Nanocrystalline Suspension

| | | Single dose mg/capsule | |
|---|---|---|---|
| | | Prescription | Prescription |
| Component | | 73 | 74 |
| Active | Apalutamide | 60 | 60 |
| ingredient | | | |
| Spatial | Hydroxypropyl cellulose | 12 | / |
| stabilizer | Hydroxypropyl | / | 12 |
| | methylcellulose acetate | | |
| | succinate | | |
| Ionic | Sodium dodecyl sulfate | 12 | 12 |
| surfactant | | | |
| Ball milling | Purified water | 50 times of | / |
| medium | | the API | |
| | | prescription | |
| | | volume | |
| | pH 6.8 medium | / | 50 times of |
| | | | the API |
| | | | prescription |
| | | | volume |

According to Table 50, the prescribed amount of spatial stabilizer and sodium dodecyl sulfate were weighed and added to the ball mill medium (purified water/pH 6.8 medium) and stirred until the solution was clear, and then the prescribed amount of apalutamide was added and the mixture was stirred to obtain the suspension; the nanocrystalline suspension of different particle sizes was collected by using NETZSCH ball mill with 0.3 mm size beads and ball milling for different times, and the particle size data are shown in Tables 51 and 52.

TABLE 51
Particle Size Distribution

| Prescription number | D10 (μm) | D50 (μm) | D90 (μm) |
|---|---|---|---|
| Prescription 73-1 | 1.016 | 2.234 | 4.423 |
| Prescription 73-2 | 0.643 | 0.902 | 2.151 |
| Prescription 73-3 | 0.459 | 0.712 | 1.562 |
| Prescription 73-4 | 0.354 | 0.518 | 0.841 |
| Prescription 73-5 | 0.302 | 0.413 | 0.583 |

TABLE 52
Particle Size Distribution

| Prescription number | D10 (μm) | D50 (μm) | D90 (μm) |
|---|---|---|---|
| Prescription 74-1 | 1.102 | 2.221 | 4.398 |
| Prescription 74-2 | 0.693 | 0.981 | 2.185 |
| Prescription 74-3 | 0.461 | 0.728 | 1.599 |
| Prescription 74-4 | 0.363 | 0.541 | 0.865 |
| Prescription 74-5 | 0.301 | 0.401 | 0.577 |

### Example 48: Dissolution Test

Prescriptions 73 and 74 of different particle sizes of ball milling fluid were put into 900 mL of pH 6.8 medium, paddle method 50 rpm, dissolution was determined by in-situ optical fiber, and the results are shown in Table 53.

TABLE 53
Dissolution Results of Prescriptions 73 and 74
Detected Using In-Situ Optical Fiber

| Prescription | | | | | | |
|---|---|---|---|---|---|---|
| number | 5 min | 10 min | 15 min | 20 min | 30 min | 60 min |
| Prescription 73-1 | 18 | 19 | 20 | 19 | 21 | 22 |
| Prescription 73-2 | 38 | 39 | 41 | 42 | 44 | 45 |
| Prescription 73-3 | 42 | 44 | 50 | 56 | 57 | 56 |
| Prescription 73-4 | 49 | 55 | 65 | 65 | 65 | 65 |
| Prescription 73-5 | 52 | 56 | 66 | 68 | 69 | 69 |
| Prescription 74-1 | 21 | 22 | 21 | 21 | 22 | 22 |
| Prescription 74-2 | 29 | 33 | 36 | 38 | 41 | 42 |
| Prescription 74-3 | 48 | 50 | 55 | 58 | 59 | 60 |
| Prescription 74-4 | 51 | 55 | 63 | 65 | 68 | 69 |
| Prescription 74-5 | 54 | 58 | 64 | 68 | 71 | 72 |
| Comparative | 7 | 8 | 10 | 11 | 12 | 15 |
| prescription 1 | | | | | | |

Ball milling was carried out with a better prescription to obtain nanocrystalline suspensions of different particle sizes, and the dissolution was determined, the inventors were pleasantly surprised to find that when the particle size of the suspension reached a certain particle size, the dissolution increased significantly, the dissolution was more than 4 times that of the ordinary preparation, the dissolution increased greatly when D50 was less than 0.7 μm, and the dissolution effect was better when D50 was less than 0.5 μm.

The scheme of the present invention has been described by a better embodiment, and it is obvious that the relevant person can make changes or appropriate changes and combinations to the method and application described herein within the content and scope of the present invention to realize and apply the present invention. Those skilled in the art may refer to the content of this article to appropriately improve the process parameters to realize the present invention. In particular, it should be noted that all such substitutions and alterations are obvious to those skilled in the art and are considered to be included in the invention.

1.-11. (canceled)

12. A composition, characterized in that it comprises a nanocrystalline composition, the nanocrystalline composition comprises an active ingredient and a stabilizer, the active ingredient is apalutamide or pharmacologically acceptable salt thereof.

13. The composition according to claim 12, the stabilizer comprises an ionic surfactant and a spatial stabilizer.

14. The composition according to claim 13, the ionic surfactant comprises at least one of sodium dodecyl sulfate, sodium dodecyl sulfonate, sodium dioctyl sulfosuccinate, benzethonium chloride, sodium docusate, lecithin, and arginine hydrochloride; or the ionic surfactant is sodium dodecyl sulfate, sodium docusate or lecithin; and/or the spatial stabilizer comprises at least one of hydroxypropyl cellulose, polyvinylpyrrolidone, vinyl pyrrolidone-vinyl acetate copolymer, polyvinyl alcohol, hydroxypropyl methylcellulose, hydroxypropyl methylcellulose acetate succinate, hydroxymethyl cellulose, chitosan, gelatin, carrageenan and cyclodextrin; or the spatial stabilizer is at least one of hydroxypropyl cellulose, vinyl pyrrolidone-vinyl acetate copolymer, polyvinyl alcohol, hydroxypropyl methylcellulose, hydroxypropyl methylcellulose acetate succinate.

15. The composition according to claim 13, the active ingredient is based on apalutamide, and the mass ratio of the ionic surfactant to the active ingredient is 1:2000-1:2; or the mass ratio of the ionic surfactant to the active ingredient is 1:200-1:2; and/or the active ingredient is based on apalutamide, and the mass ratio of the spatial stabilizer to the active ingredient is 1:30-2:1; or the mass ratio of the spatial stabilizer to the active ingredient is 1:15-2:1.

16. The composition according to claim 14, the active ingredient is based on apalutamide, and the mass ratio of the ionic surfactant to the active ingredient is 1:2000-1:2; or the mass ratio of the ionic surfactant to the active ingredient is 1:200-1:2; and/or the active ingredient is based on apalutamide, and the mass ratio of the spatial stabilizer to the active ingredient is 1:30-2:1; or the mass ratio of the spatial stabilizer to the active ingredient is 1:15-2:1.

17. The composition according to claim 12, the composition also comprises a protective agent.

18. The composition according to claim 17, the protective agent comprises at least one of mannitol, lactose, fructose, glycine, glucose, sucrose, maltose, trehalose, sorbitol, xylitol, polydextrose, fructooligosaccharides, malto-oligosaccharides, galacto-oligosaccharides, dextrin, gum arabic, microcrystalline cellulose, anhydrous calcium hydrogen phosphate, pregelatinized starch and corn starch.

19. The composition according to claim 17, the active ingredient is based on apalutamide, the mass ratio of the protective agent to the active ingredient is 8:1-1:8; or the mass ratio of the protective agent to the active ingredient is 8:1-1:4; or the mass ratio of the protective agent to the active ingredient is 8:1-1:2.

20. The composition according to claim 18, the active ingredient is based on apalutamide, the mass ratio of the protective agent to the active ingredient is 8:1-1:8; or the mass ratio of the protective agent to the active ingredient is 8:1-1:4; or the mass ratio of the protective agent to the active ingredient is 8:1-1:2.

21. The composition according to claim 12, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

22. The composition according to claim 15, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

23. The composition according to claim 16, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

24. The composition according to claim 17, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

25. The composition according to claim 18, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

26. The composition according to claim 19, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

27. The composition according to claim 20, the active ingredient is based on apalutamide, and the content of the active ingredient in the non-volatile components of the nanocrystalline composition is 10 wt %-95 wt %; and/or the active ingredient is based on apalutamide, and the content of the active ingredient in the non-volatile components of the composition is 10 wt %-40 wt %; or the content of the active ingredient in the non-volatile components of the composition is 15 wt %-25 wt %.

28. The composition according to claim 12, the particle size D90 of the nanocrystalline composition is 1 nm-1000 nm; and/or the particle size D50 of the nanocrystalline composition is 900 nm or less.

29. The composition according to claim 12, the nanocrystalline composition is prepared using a method comprising at least one method selected from a precipitation method, an emulsification method, a high-pressure homogenization method and a medium milling method; or the nanocrystalline composition is prepared by a medium milling method.

30. The method for preparing the composition of claim 12 comprises the following steps:
(1) Stabilizer and apalutamide are mixed with water to obtain a suspension;
(2) The suspension is mixed with a milling medium and milled to obtain a nanocrystalline composition;
(3) The nanocrystalline composition is optionally added with pharmaceutically acceptable excipients, mixed, to obtain a composition; or the nanocrystalline composition is added with a protective agent, cured by a curing process, optionally added with at least one of filler, disintegrant, glidant or lubricant, mixed, to obtain a composition.

31. The method for preparing the composition of claim 27 comprises the following steps:
(1) Stabilizer and apalutamide are mixed with water to obtain a suspension;
(2) The suspension is mixed with a milling medium and milled to obtain a nanocrystalline composition;
(3) The nanocrystalline composition is optionally added with pharmaceutically acceptable excipients, mixed, to obtain a composition; or the nanocrystalline composition is added with a protective agent, cured by a curing process, optionally added with at least one of filler, disintegrant, glidant or lubricant, mixed, to obtain a composition.
